SEQUENCE LISTING (10 sequences)

SEQ ID NO: 1 - DNA, 462 bp, Solanum lycopersicum
tgtagtattctagttaaactacctttgaatgtctagtaccagacttataatagtattttggtagaatgtctgcgtgtatatttagttgggtgaatggactggtgatctgcttatagacttggggaatcttcccctcgacccctcacgagctagcttttggggttgcatatcattgtccagctaaccaaactaaactatatgtatttaaaccatatatgaagtataccttatgggaaatttgaaggaatgaaggttattactagtatgttaaactcatgcttatgtaactttgagcattaacacataaaaaggggcagcgcggtgcactatattctccggctataactagggacattgaacttgcatttttgcgagagactatttaaatggctagagacaaaggcacgttgccataatggacataaattaacgactatatacaaagttagccatgtgtcacga

SEQ ID NO: 2 - DNA, 472 bp, Solanum lycopersicum
ttttttttatttgttggaaagaattggcttttgaatatcaagaattattatcttattattattcctctccatttttaattaaatggagggggtatctaataatgttcatggagttatattagcaatatcatccagtatttttattggttccagctttgttattaaaaaacaaggtctaaagaaggctggtgcaaatggaaaaagagcaggtttattctttttctcgcgctatgattcaaaaactcgatgaataataggcttgcatctttctccccttaaataccagacttttgtattcagagaatcgtgacacatggctaactttgtatatagtcgttaatttatgtccattatggcaacgtgcctttgtctctagccatttaaatagtctctcgcaaaaatgcaagttcaatgtccctagttatagccggagaatatagtgcaccgcgctgcccctttttatgtgttaatg

SEQ ID NO: 3 - DNA, 495 bp, Solanum lycopersicum
taaatctgttgaaatagctgcaaatctactcgtgcaagaaggtacgcgtttacattggttgagggaggacatctagagaaatgagacacattcgatcgtatgtatacgatgcaaaggcaaaggaagttggaggtcaaaaacctattacaagatattcaacaactggcaggtgatgtggaggatctattagatgagttccttccaaaaattcaacaatccaataaggtcatttgatgccttaagactgtttcttttgcggataagtttgctatggagattgagaacataaaaagaagagttgctgatattgaccgtgtaaggacaacttaccacatcacggatactagtaacaataatgatgattgcattccaatggaccagagaagacgattccttcatgctaatgatgaaacagaggtcatcggattggatcatgacttcaacaagctccaacacaaattgcttcttcaagatttgccttacggagttgtttca

SEQ ID NO: 4 - DNA, 543 bp, Solanum lycopersicum
ggagacagcttgcatgcctgcagtcaccacaatcgctagcggtatacctccacatctctcaactatacatctaccaatatttaccaagtctggtgaagtattggcccaattatcatcaacaaaattacagattttcttggtaaagagttcaaaactgttctctgagtctagaggttgcaactcgtggattgagaaatcccctcctatgtatctgcctacattactatttcgagaggttataattatcctactgccaatttttgaatacgattcaggaaggacgagttttagatcatcccaaatttcaacatcccaaatgtcatctaaaaggataacgtacctttttattttcaagagtgatcttaggttgtgttccaagttctctttcctttcttcttccgtcagtccaacttgtttggctatgttaagcaagatttctcccgcccttggctgctgcgagacatagaccaatccagaacattcaaattgatgacggacgtgcctataaagtttcttggcaagagtagtttttcccaaaccggg

SEQ ID NO: 5 - DNA, 23 bp, Solanum lycopersicum
ggtgatctgcttatagacttggg

SEQ ID NO: 6 - DNA, 22 bp, Solanum lycopersicum
aaggtctaaagaaggctggtgc

SEQ ID NO: 7 - DNA, 23 bp, Solanum lycopersicum
aaggcaaaggaagttggaggtca

SEQ ID NO: 8 - DNA, 24 bp, Solanum lycopersicum
agaggttgcaactcgtggattgag

SEQ ID NO: 9 - DNA, 20 bp, Solanum lycopersicum
gacttgttgccataggttcc

SEQ ID NO: 10 - DNA, 20 bp, Solanum lycopersicum
gcccaccccgaagttaatcc

(19) United States
(12) Patent Application Publication
(10) Pub. No.: US 2012/0054905 A1
(43) Pub. Date: Mar. 1, 2012

(54) Marker Assisted Selection for Coupling Phase Resistance to Tomato Spotted Wilt Virus and Late Blight in Tomato

(75) Inventors: David M. Francis, Wooster, OH (US); Matthew D. Robbins, Hyrum, UT (US); Dilip R. Panthee, Fletcher, NC (US); Randolph G. Gardner, Mills River, NC (US); Mikel R. Stevens, Orem, UT (US)

(73) Assignee: THE OHIO STATE UNIVERSITY, Columbus, OH (US)

(21) Appl. No.: 12/869,345

(22) Filed: Aug. 26, 2010

(51) Int. Cl. A01H 1/02; A01H 5/00

(52) U.S. Cl. 800/265; 800/317.4

(57) ABSTRACT

A Solanum lycopersicum plant including within its genome at least one Tomato Spotted Wilt Virus (TSWV) resistance allele and at least one Phytophthora infestans resistance allele. The resistance alleles are present in the coupling phase at different loci on one chromosome and the plant is resistant against TSWV and resistant against at least Phytophthora infestans. A method for producing a hybrid Solanum lycopersicum plant including (a) obtaining a Solanum lycopersicum plant having within its genome at least one Tomato Spotted Wilt Virus (TSWV) resistance allele and at least one Phytophthora infestans resistance allele in the coupling phase; and (b) crossing the Solanum lycopersicum plant with a second Solanum lycopersicum plant bearing an additional resistance allele.

### FIELD OF THE INVENTION

The present invention relates to methods for combining (pyramiding) tightly-linked genes of commercial importance in tomato (Solanum lycopersicum formerly Lycopersicon esculentum). Specifically, the invention relates to creating tomato plants comprising an effective Tomato Spotted Wilt Virus resistance gene (Sw-5) and an effective Phytophthora infestans resistance gene (Ph-3) in coupling phase (in cis), such that these closely linked genes are co-inherited as if a single unit.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

There are over two hundred documented diseases of cultivated tomato [Compendium of Tomato Diseases. J. B. Jones, J. P. Jones, R. E. Stall, T. A. Zitter, editors, (1997) American Phytopathological Society Press, St. Paul, Minn.]. Thus, growers may employ an integrated pest management strategy including both cultural practices and pesticide use to combat the damage caused by these pathogens. An example of a cultural practice is the use of netting over tomato plants, which provides a physical barrier that can be effective in excluding disease-bearing insects from infecting the crop.

Despite numerous research studies regarding transgenic approaches against diseases of plants, there are currently no transgenic tomato varieties available to the grower that are resistant to any pathogens. Further, there remains an issue of public resistance, which, combined with the high cost of obtaining regulatory approval, has effectively prohibited this promising technology from being used in commercial tomato cultivation.

Introgression of disease resistance genes into modem cultivars using traditional breeding approaches has remained an effective technology available for combating the majority of plant diseases. Because of its continued success, the approach is still a primary focus in both academic and commercial tomato breeding programs. Among the hundreds of tomato pathogens, diseases caused by the Tomato Spotted Wilt Virus (TSWV) and Phytophthora infestans are among the most important to the commercial grower.

TSWV is a Tospovirus. The Tospoviruses are a group of plant infecting negative RNA viruses found within the family Bunyaviridae. Each virus has a single stranded RNA genome with negative polarity—classified as a Class V virus [(−)ssRNA viruses)]. The genome is linear and is 17.2 kb in size. It is segmented into three segments termed S (2.9 kb), M (5.4 kb) and L (8.9 kb). The S and M RNA segments encode for proteins in an ambisense (both sense and antisense) orientation.

Tospoviruses are naturally vectored by a thrips, which are tiny, slender insects with fringed wings and include thunderflies, thunderbugs, storm flies, western flower thrips, tobacco thrips, onion thrips and corn lice. Infection with the virus results in spotting and wilting of the plant, reduced vegetative and fruit output, and often eventual death. Early symptoms of infection by TSWV may be difficult to diagnose. In young infected plants, the characteristic symptoms include inward cupping of leaves and leaves that develop a bronze cast followed by dark spots. As the infection progresses additional symptoms develop which include dark streaks on the main stem and wilting of the top portion of the plant. Fruit may be deformed, show uneven ripening and often have raised bumps and necrotic lesions on the surface. Further symptoms include stunted growth, necrotic patches, and chlorotic ringspots. Once a plant becomes infected, the disease cannot be controlled. No antiviral cures have been developed for plants infected with a Tospovirus, and infected plants are generally removed from a field and destroyed in order to prevent the spread of the disease.

TSWV is prevalent in warm climates in regions with a high population of thrips, and is an agricultural pest in Asia, Americas, Europe, and Africa. Over the past several years, outbreaks of disease caused by TSWV have become more prevalent in these regions. Commercial tomato production has shifted over the past twenty-five years from temperate to more tropical growing regions in the world, due to lower labor costs, better transportation and treaties like the North American Free Trade Agreement and the World Trade Organization Agreement. This shift in geography coincides with the problem, discussed above, that TSWV is prevalent in warm climates in regions with a high population of thrips. Thus, as tomato growing regions have shifted towards tropical climates, the disease caused by TSWV has become more pronounced.

Apart from TSWV, diseases in tomato may also be caused by Phytophthora infestans. Phytophthora infestans is a species of oomycete (or water mold) that causes the serious disease known as late blight. Late blight can have devastating effects by destroying entire crops. Spores of the mold develop on the leaves, spreading through the crop when temperatures are above 10° C. (50° F.) and humidity is over 75% for 2 days or more. Rain can wash spores into the soil where they infect other plants. Spores can also travel long distances on the wind.

The early stages of late blight are easily missed, and not all plants are affected at once. Symptoms include the appearance of dark blotches/lesions on leaf tips and plant stems. White mold will appear under the leaves in humid conditions and the whole plant may quickly collapse.

Despite chemical options to control Phytophthora infestans, it is still difficult to manage due to its rapid spread. Around the world, the disease causes billions of dollars of damage to crops each year.

Chemical control of thrips (spreading TSWV) and Phytophthora infestans can be effective, but the misuse of pesticides in commercial tomato production has resulted in pesticide-resistant thrips that can vector TSWV. Thrips are very difficult to control with even effective pesticides. Chemical control is “lackluster” at its very best, and even with systemic insecticides it takes time to kill the thrips. This time lag is often sufficient for them to have passed the virus along. Finally, thrips migrate from surrounding areas and continue to pass along this ubiquitous virus. Further, chemical options for control of Phytophthora infestans and chemical options for control of TSWV are being reduced, for reasons ranging from insect vectors becoming resistant to the pesticide to public health concerns over the use of pesticides. At the same time, new chemical controls are becoming available; however, these may suffer the same drawbacks as previous chemical controls (i.e., resistance, public concern, etc.). The high development costs attendant to producing transgenic resistant cultivars is likewise an impediment to the development of TSWV and Phytophthora infestans-resistant cultivars using a genetic engineering approach. Those skilled in the art will thus recognize the need for alternatives to these chemical control strategies and transgenic strategies for the control of TSWV, Phytophthora infestans, and other causes of plant diseases in tomato production. The introgression of naturally occurring resistance genes remains the most effective option for controlling tomato pathogens today.

Although the inheritance of a resistance phenotype can be quantitative and polygenic, it is common in plants to have simply inherited resistance with dominant, semi-dominant, or even recessive modes of action controlled by individual single loci. Plant resistance genes often encode proteins that act as receptors that bind specific pathogen-encoded ligands (receptor-ligand model) or recognize plant proteins that are modified by pathogen effectors (guard model). This pathogen-specific recognition and subsequent response by the plant is a phenomena first described by Flor in the late 1940's and referred to as ‘gene-for-gene’ resistance, [reviewed by Flor (1971) Ann. Review of Phytopathology 9:275-296]. This specific plant protein-pathogen protein combination triggers a signal transduction pathway that ultimately results in a resistance phenotype [Baker et al. (1997), Science 276:726-733; Staskawicz et al. (1995) Science 268:661-667]. In response to recognition of pathogen attack, the host can respond with a strengthening of the cell wall, an oxidative burst, induction of defense gene expression and rapid cell death at the infection site called the hypersensitive response.

For most breeding objectives, commercial breeders work with germplasm often referred to as the ‘cultivated type’. This germplasm is easier to breed with because it generally performs well when evaluated for horticultural/agronomic performance. However, the performance advantage the cultivated types provide is often offset by a lack of allelic diversity. This is the trade-off a breeder accepts when working with cultivated germplasm—better overall performance, but a lack of allelic diversity. Breeders generally accept this trade-off because progress is faster when working with cultivated material than when breeding with genetically diverse sources.

In contrast, when a breeder makes either wide intra-specific crosses or inter-specific crosses, a converse trade-off occurs. In these examples, a breeder typically crosses cultivated germplasm with a non-cultivated type. In such crosses, the breeder can gain access to novel alleles from the non-cultivated type, but has to overcome the genetic drag associated with the donor parent. Besides the difficulty of negative traits associated with the donor parent, this approach often fails because of fertility or fecundity problems.

There are many wild relatives that can be crossed with cultivated tomato, including S. pennellii, S. habrochaites, S. peruvianum, S. chilense, S. neorickii, S. chmielewskii, S. cheesmanii, and S. pimpinellifolium. The genetic distance between the wild species and the cultivated S. lycopersicum correlates with the difficulty of both making the inter-specific cross, and successfully creating a new commercial cultivar with an added trait [Genetics and breeding. M A Stevens and C M Rick. In: The tomato crop: A scientific basis for improvement. J G Atherton and J Rudich, editors. Chapman and Hall, (1994), London]. For example, species like S. pimpinellifolium, S. cheesmanii, S. chmielewskii, and S. neorickii are the easiest wild species to use as donors for trait introgression into the modern tomato. In contrast, S. pennellii, S. chilense, S. habrochaites, and S. peruvianum are far more difficult species for trait introgression into the modern tomato [ibidem]. When using these more distantly related species, it is not uncommon to have to use bridging species and embryo rescue for early generation crosses. Even with these extra steps, one can face significant segregation distortion, fertility problems, reduced recombination, and genetic drag. Even in advanced generations, a suppression of recombination in the introgressed area of the genome presents the primary obstacle to reducing the genetic drag enough to create a successful commercial cultivar.

Sw-5 and Ph-3 have been tightly linked to the restriction fragment length polymorphism (RFLP) markers CT220 and TG591A respectively [Brommonschenkel, S. H., A. Frary, and S. D. Tanksley. 2000. The broad-spectrum tospovirus resistance gene Sw-5 of tomato is a homolog of the root-knot nematode resistance gene Mi. Mol. Plant-Microbe Interact. 13:1130-1138; Chunwongse, J., C. Chunwongse, L. Black, and P. Hanson. 2002. Molecular mapping of the Ph-3 gene for late blight resistance in tomato. J. Hort. Sci. Biotechnol. 77:281-286; Chunwongse, J., C. Chunwongse, L. L. Black, and P. M. Hanson. 1998. Mapping of the Ph-3 gene for late blight from L. pimpinellifolium L3708. Rep. Tomato Genet. Coop. 48:963-971; Folkertsma, R. T., M. I. Spassova, M. Prins, M. R. Stevens, J. Hille, and R. W. Goldbach. 1999. Construction of a bacterial artificial chromosome (BAC) library of Lycopersicon esculentum cv. Stevens and its application to physically map the Sw-5 locus. Mol. Breed. 5:197-207; Stevens, M. R., E. M. Lamb, and D. D. Rhoads. 1995. Mapping the Sw-5 locus for Tomato spotted wilt virus resistance in tomatoes using RAPD and RFLP analyses. Theor. Appl. Genet. 90:451-456]. These two markers are near the telomere of the long arm of chromosome 9. Furthermore, CT220 and TG591A have been found to be 5 cM (centiMorgans) or less apart on the tomato maps EXPEN 2000 and the EXPEN 1992 respectively (http://sgn.cornell.edu; Tanksley et al. 1992). Since Sw5 and Ph3 originate from different wild relatives of tomato they are naturally in genetic repulsion phase (in trans). Also, genetic evidence suggests the genes are close to each other on chromosome 9. And so the phase and short genetic distance between these genes provides a potential challenge to recombining them into the coupling phase (in cis) such that they are inherited together from the same parent. An additional challenge is that a number of tomato breeding programs and studies have identified crossover suppression in chromosomal areas of cultivated tomatoes which are derived from wild relatives [Ganal, M. W. and S. D. Tanksley. 1996. Recombination around the Tm2a and Mi resistance genes in different crosses of Lycopersicon peruvianum. Theor. Appl. Genet. 92:101-108; Liharska, T., M. Koornneef, M. vanWordragen, A. vanKammen, and P. Zabel. 1996. Tomato chromosome 6: Effect of alien chromosomal segments on recombinant frequencies. Genome 39:485-491; Zamir, D. and Y. Tadmor. 1986. Unequal segregation of nuclear genes in plants. Bot. Gaz. 147:355-358].

Thus, even though one may identify a useful trait in a wild species and target that trait for introgression into the cultivated species, there is no guarantee of success. Most successful commercial tomato breeders work their entire careers without successfully completing an introgression from a wild species to create commercial cultivars. The barriers to success include segregation distortion, which may result in areas of the wild genome that can be difficult to impossible to introgress. Further, some of the wild species of the modern tomato are self-incompatible, meaning that they cannot self pollinate. As a result of the self-incompatibility, these plants are highly heterogeneous, having different alleles at many loci. The highly heterogeneous nature of these wild species can also hinder the introgression of the most efficacious allele of interest.

Further, tomato breeders are faced with a limitation in their ability to deliver multiple resistance genes that map to the telomeric region of the long arm of chromosome 9 while retaining the ability to mask the genetic drag associated with these introgressions. To pyramid all the known resistance genes that map in this region of chromosome 9 in a hybrid cultivar, a breeder would have to have one parent with the introgression from S. pimpinellifolium containing the Phytophthora infestans resistant gene Ph-3, another parent with the introgression from S. peruvianum containing the TSWV resistance gene Sw-5, and another parent with an introgression from another tomato species containing an additional resistance gene, and yet another parent containing the ‘+’ type alleles from S. lycopersicum in order to mask the genetic drag associated with some of these introgressions. This task is very difficult for the breeder because they have only two parent lines to choose from to make hybrid cultivars. An example of such a dilemma is also shown graphically by Ho et al. [(1992) The Plant Journal 2:971-982, see FIG. 6], and by Liharska et al. [(1996) Genome 39:485-491, see FIG. 1].

Thus, there remains a need to identify a recombinational event in this area of the genome known to have severely suppressed recombination, and that will contain the most efficacious allele for TSWV resistance, Sw-5, originally introgressed from S. peruvianum, with the most efficacious allele for Phytophthora infestans resistance, Ph-3, originally introgressed from S. pimpinellifolium. Tightly linked alleles juxtapositioned in this manner are said to be in the coupling phase, or in cis. Such a combination of efficacious resistance alleles in cis would allow tomato breeders to create tomato hybrids with the most efficacious resistance to TSWV and Phytophthora infestans, while retaining the freedom of having a second inbred parent to either mask the genetic drag, or deliver additional resistance genes, or other (Ph-1, Ph-2, Ph-4, Ph-5, Sw-7) or yet to be discovered resistance alleles in this disease cluster.

### SUMMARY OF INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

As described above, although variety development for multiple disease resistance in tomato (Solanum lycopersicum) is a desirable goal, the process is often challenging. This is largely due to the need of large-scale screening and lack of available resistant material with cultivated genetic background. It is often further complicated by linkage drag of unacceptable characteristics tightly linked with resistance, emergence of new disease pathogens, and the necessity of selecting for resistance to multiple diseases [Yang, W. C. and D. M. Francis. 2005. Marker-assisted selection for combining resistance to bacterial spot and bacterial speck in tomato. J. Amer. Soc. Hort. Sci. 130:716-721]. Marker-assisted selection (MAS) offers an opportunity to overcome some of the problems associated with phenotypic selection and to the ability to combine resistances genes.

As is known to those of ordinary skill in the art, MAS is a process whereby a marker (e.g., one based on protein or DNA/RNA variation) is used for selection of a genetic determinant or determinants of a trait of interest (e.g., disease resistance). Thus, in MAS, a trait of interest is selected not based on the trait itself but on a marker linked to it. For example, if MAS is being used to select individuals with a disease, the level of disease is not quantified but rather a marker allele which is linked with disease is used to determine presence of disease resistance. The assumption is that the linked marker allele associates with the gene and/or quantitative trait locus (QTL—a stretch of DNA that is closely linked to the gene or genes that underlie a trait with quantitative inheritance) of interest.

Generally, the first step of MAS is to map the gene or QTL of interest based on the statistical association of the trait with a specific DNA polymorphism, and then use this information for marker assisted selection. Generally, the markers to be used should be close to gene of interest (<5 recombination units or cM) in order to reduce linkage drag and ensure that only a minor fraction of the selected individuals will be recombinants. Generally, not only a single marker but rather two markers are used in order to reduce the chances of an error due to homologous recombination. For example, if two flanking markers are used at same time with an interval between them of approximately 20 cM, there is higher probability for recovery of the target gene (Frisch, M., M. Bohn and A. A. Melchinger. 1999. Minimum Sample Size and Optimal Positioning of Flanking Markers in Marker-Assisted Backcrossing for Transfer of a Target Gene. Crop Sci 39:967-975; ERRATA Frisch et al. 39 (6): 1903. (1999); Hospital, F. and A. Charcosset. 1997. Marker assisted introgression of quantitative trait loci. Genetics 147:1469-1485.). The Sw-5 and Ph-3 alleles have been previously mapped and markers are known.

In plants, QTL mapping is generally achieved using bi-parental cross populations (i.e., a cross between two parents which have a contrasting phenotype for the trait of interest are developed). Commonly used populations are recombinant inbred lines (RILs), doubled haploids (DH), back cross, and F₂. An F₂ population is used in the example herein. However, those of ordinary skill in the art will recognize that other populations may be used.

As described above, in one aspect of the present invention, two important simply inherited disease resistance genes have been introgressed into cultivated tomato from wild tomato relatives: Sw-5 for TSWV and Ph-3 for late blight. Both diseases are responsible for substantial tomato crop losses worldwide [Foolad, M. R., H. L. Merk, and H. Ashrafi. 2008. Genetics, genomics and breeding of late blight and early blight resistance in tomato. Crit. Rev. Plant Sci. 27:75-107; Fry, W. E. and S. B. Goodwin. 1997. Re-emergence of potato and tomato late blight in the United States. Plant Dis. 81:1349-1357; Kim, M. J. and M. A. Mutschler, 2005. Transfer to processing tomato and characterization of late blight resistance derived from Solanum pimpinellifolium L. L3708. J. Amer. Soc. Hort. Sci. 130:877-884; Kim, M. J. and M. A. Mutschler. 2006. Characterization of late blight resistance derived from Solanum pimpinellifolium L3708 against multiple isolates of the pathogen Phytophthora infestans. J. Amer. Soc. Hort. Sci. 131:637-645; Mumford, R. A., I. Barker, and K. R. Wood. 1996. The biology of the Tospoviruses. Ann. Appl. Biol. 128:159-183; Roselló, S., M. J. Díez, and F. Nuez. 1996. Viral diseases causing the greatest economic losses to the tomato crop. I. The Tomato spotted wilt virus—a review. Sci. Hort. 67:117-150]. Several resistance genes have been identified for both TSWV and late blight; however, these two genes (Sw-5 and Ph-3) have been found to be especially economically valuable for their broad level of resistance to each of these diseases [Foolad, M. R., H. L. Merk, and H. Ashrafi. 2008. Genetics, genomics and breeding of late blight and early blight resistance in tomato. Crit. Rev. Plant Sci. 27:75-107; Gordillo, L. F., M. R. Stevens, M. A. Millard, and B. Geary. 2008. Screening two Lycopersicon peruvianum collections for resistance to Tomato spotted wilt virus. Plant Dis. 92:694-704].

More specifically, in this aspect of the present invention, the TSWV resistance gene Sw-5 was introgressed from S. peruvianum (previously Lycopersicon peruvianum Mill.; accession unknown) [Stevens, J. M. 1964. Tomato breeding; Dept. Agricultural Technical Services. Republic of South Africa, Project Report W-Vv1; Stevens, M. R., S. J. Scott, and R. C. Gergerich. 1992. Inheritance of a gene for resistance to Tomato spotted wilt virus (TSWV) from Lycopersicon peruvianum Mill. Euphytica 59:9-17; van Zijl, J. J. B., S. E. Bosch, and C. P. J. Coetzee. 1986. Breeding tomatoes for processing in South Africa. Acta Hort. 194:69-75]. This gene has been identified to be resistant to the Tospovirus species TSWV, GRSV (Groundnut ring spot virus), and TCSV (Tomato chlorotic spot virus) [Bioteux, L. S., T. Nagata, and L.d.B. Giordano. 1993. Field resistance of tomato Lycopersicon esculentum lines to tomato spotted wilt disease. Rep. Tomato Genet. Coop. 43:7-9; Stevens, M. R., S. J. Scott, and R. C. Gergerich. 1992. Inheritance of a gene for resistance to Tomato spotted wilt virus (TSWV) from Lycopersicon peruvianum Mill. Euphytica 59:9-17; van Zijl, J. J. B., S. E. Bosch, and C. P. J. Coetzee. 1986. Breeding tomatoes for processing in South Africa. Acta Hort. 194:69-75]. The late blight resistance Ph-3, derived from S. pimpinellifolium L., is of incomplete dominant inheritance; however, it has demonstrated strong resistance to a spectrum of Phytophthora infestans isolates [Chunwongse, J., C. Chunwongse, L. Black, and P. Hanson. 2002. Molecular mapping of the Ph-3 gene for late blight resistance in tomato. J. Hort. Sci. Biotechnol. 77:281-286; Chunwongse, J., C. Chunwongse, L. L. Black, and P. M. Hanson. 1998. Mapping of the Ph-3 gene for late blight from L. pimpinellifolium L3708. Rep. Tomato Genet. Coop. 48:963-971; Foolad, M. R., H. L. Merk, and H. Ashrafi. 2008. Genetics, genomics and breeding of late blight and early blight resistance in tomato. Crit. Rev. Plant Sci. 27:75-107].

Sw-5 and Ph-3 have been tightly linked to the restriction fragment length polymorphism (RFLP) markers CT220 and TG591A respectively [Brommonschenkel, S. H., A. Frary, and S. D. Tanksley. 2000. The broad-spectrum tospovirus resistance gene Sw-5 of tomato is a homolog of the root-knot nematode resistance gene Mi. Mol. Plant-Microbe Interact. 13:1130-1138; Chunwongse, J., C. Chunwongse, L. Black, and P. Hanson. 2002. Molecular mapping of the Ph-3 gene for late blight resistance in tomato. J. Hort. Sci. Biotechnol. 77:281-286; Chunwongse, J., C. Chunwongse, L. L. Black, and P. M. Hanson. 1998. Mapping of the Ph-3 gene for late blight from L. pimpinellifolium L3708. Rep. Tomato Genet. Coop. 48:963-971; Folkertsma, R. T., M. I. Spassova, M. Prins, M. R. Stevens, J. Hille, and R. W. Goldbach. 1999. Construction of a bacterial artificial chromosome (BAC) library of Lycopersicon esculentum cv. Stevens and its application to physically map the Sw-5 locus. Mol. Breed. 5:197-207; Stevens, M. R., E. M. Lamb, and D. D. Rhoads. 1995. Mapping the Sw-5 locus for Tomato spotted wilt virus resistance in tomatoes using RAPD and RFLP analyses. Theor. Appl. Genet. 90:451-456]. These two markers are near the telomere of the long arm of chromosome 9. Furthermore, CT220 and TG591A have been found to be 5 cM (centiMorgans) or less apart on the tomato maps EXPEN 2000 and the EXPEN 1992 respectively [http://sgn.cornell.edu; Tanksley et al. 1992]. Since these two genes originate from different wild relatives of tomato they are naturally in genetic repulsion phase (in trans). Also, these genes are very close to each other on chromosome 9. And so the phase and short genetic distance between these genes provides a potential challenge to recombining them into the coupling phase (in cis), as described in the Background section. An additional challenge is that a number of tomato breeding programs and studies have identified crossover suppression in cultivated tomatoes chromosomal areas derived from wild relatives [Ganal, M. W. and S. D. Tanksley. 1996. Recombination around the Tm2a and Mi resistance genes in different crosses of Lycopersicon peruvianum. Theor. Appl. Genet. 92:101-108; Liharska, T., M. Koornneef, M. vanWordragen, A. vanKammen, and P. Zabel. 1996. Tomato chromosome 6: Effect of alien chromosomal segments on recombinant frequencies. Genome 39:485-491; Zamir, D. and Y. Tadmor. 1986. Unequal segregation of nuclear genes in plants. Bot. Gaz. 147:355-358].

In another aspect of the present invention, since both TSWV and late blight can be extremely devastating, the development of tomato germplasm homozygous for Sw-5 and Ph-3 enables the possibility of pyramiding Ph-1, Ph-2, Ph-3, Ph-4, and Ph-5 with Sw-5 and Sw-7 for broader resistance than presently available [Dockter, K. G., D. S. O'Neil, D. L. Price, J. Scott, and M. R. Stevens. 2009. Molecular mapping of the Tomato spotted wilt virus resistance gene Sw-7 in tomato. HortScience 44:1123-1123; Foolad, M. R., H. L. Merk, and H. Ashrafi. 2008. Genetics, genomics and breeding of late blight and early blight resistance in tomato. Crit. Rev. Plant Sci. 27:75-107; Gordillo, L. F., M. R. Stevens, M. A. Millard, and B. Geary. 2008. Screening two Lycopersicon peruvianum collections for resistance to Tomato spotted wilt virus. Plant Dis. 92:694-704; Price, D. L., J. W. Memmott, J. W. Scott, S. Olson, and M. R. Stevens. 2007. Identification of molecular markers linked to a new Tomato spotted wilt virus resistance source in tomato. Rep. Tomato Genet. Coop. 57:35; Scott, J. W., M. R. Stevens, and S. M. Olson. 2005. An alternative source of resistance to Tomato spotted wilt virus. Rep. Tomato Genet. Coop. 55:40-41]. Thus, this aspect of the present invention provides the development of lines homozygous for both genes. However, the high potential of crossover suppression and the need for screening with both disease organisms using phenotypic selection in search of plants in coupling phase for these two genes presents a considerable challenge, as will be recognized by one of ordinary skill in the art. And so, another aspect of the present invention utilizes marker assisted selection (MAS) to quickly screen a large F₂ population for unique candidate plants with crossovers placing Sw-5 and Ph-3 in coupling phase, followed by screening the candidate plants for phenotypic expression of resistance for each disease.

Thus, one aspect of the present invention provides a Solanum lycopersicum plant comprising within its genome at least one TSWV resistance allele and at least one Phytophthora infestans resistance allele, wherein the resistance alleles are present in coupling phase at different loci on one chromosome and in that the plant is resistant to TSWV and highly resistant to Phytophthora infestans.

In another illustrative embodiment, the TSWV resistance allele is the allele designated as Sw-5. In another illustrative embodiment, the Phytophthora infestans resistance allele is the allele designated as Ph-3. In a further illustrative embodiment, the TSWV resistance allele and the Phytophthora infestans resistance allele are from S. peruvianum and S. pimpinellifolium, respectively.

In the various aspects and embodiments of the present invention, the TSWV resistance allele and the Phytophthora infestans resistance may be non-transgenic.

In another aspect of the invention a fruit or a seed of such a Solanum lycopersicum plant is provided.

Another aspect of the present invention may provide an inbred commercial Solanum lycopersicum plant, or, alternatively, a plant according to this invention may be used as parent in a cross with another Solanum lycopersicum plant. The invention thus provides a hybrid Solanum lycopersicum plant produced by the method of crossing a plant of the invention with an inbred plant lacking the TSWV resistance allele and lacking the Phytophthora infestans resistance allele.

In one illustrative embodiment of this aspect of the invention, a hybrid Solanum lycopersicum plant is provided where both of the TSWV resistance allele and the Phytophthora infestans resistance allele are heterozygous. Further provided is such a hybrid plant having good horticultural characteristics, and still further provided is a hybrid plant having greatly reduced genetic drag normally associated with the wild tomato species introgressions providing the TSWV resistance allele and the Phytophthora infestans resistance allele.

The hybrid plant may show greatly reduced genetic drag effects as are those associated with the wild species S. pimpinellifolium, and greatly reduced genetic drag effects as are associated with the wild species S. peruvianum. Further, the hybrid plant may present greatly reduced genetic drag symptoms selected from the group of symptoms consisting of auto-necrosis, longer internodes, smaller fruit, less fruit set and horticulturally inferior plant architecture.

The loci of the TSWV resistance allele and the Phytophthora infestans resistance allele occur within the same disease resistance cluster on the chromosome. Thus, in yet another illustrative embodiment of the invention, at least one additional disease resistance allele within the cluster is provided in the repulsion phase, or in trans to the Sw-5 (TSWV resistance) allele and the Ph-3 (Phytophthora infestans resistance) allele. In one alternative embodiment of this aspect of the invention, the additional disease resistance allele may be chosen from Ph-1, Ph-2, Ph-4, Ph-5 and Sw-7.

### DETAILED DESCRIPTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described above, although variety development for multiple disease resistance in tomato (Solanum lycopersicum) is a desirable goal, the process is often challenging. This is largely due to the need of large-scale screening and lack of available resistant material with cultivated genetic background. It is often further complicated by linkage drag of unacceptable characteristics tightly linked with resistance, emergence of new disease pathogens, and the necessity of selecting for resistance to multiple diseases [Yang, W. C. and Francis, D. M., 2005. Marker-assisted selection for combining resistance to bacterial spot and bacterial speck in tomato. J. Amer. Soc. Hort. Sci. 130:716-721]. Marker-assisted selection (MAS) offers an opportunity to overcome some of the problems associated with phenotypic selection and to the ability to combine resistances genes.

As is known to those of ordinary skill in the art, MAS is a process whereby a marker (e.g., one based on protein or DNA/RNA variation) is used for selection of a genetic determinant or determinants of a trait of interest (e.g., disease resistance).

Thus, in MAS, a trait of interest is selected not based on the trait itself but on a marker linked to it. For example, if MAS is being used to select individuals with a disease, the level of disease is not quantified but rather a marker allele which is linked with disease is used to determine the probable level of disease resistance. The assumption is that the linked marker allele associates with the gene and/or quantitative trait locus (QTL—a stretch of DNA that is closely linked to the genes that underlie the trait in question) of interest.

Generally, the first step of MAS is to map the gene or QTL of interest by demonstrating a statistical association between marker and trait, and then using this information for marker assisted selection. Generally, the markers to be used should be close to gene of interest (<5 recombination units or cM) in order to ensure that only minor fraction of the selected individuals will be recombinants. Generally, not only a single marker but rather two markers are used in order to reduce the chances of an error due to homologous recombination. For example, if two flanking markers are used at same time with an interval between them of approximately 20 cM, there is higher probability (99%) for recovery of the target gene. Regarding Sw-5 and Ph-3: These alleles have been previously mapped and markers are known.

In plants, QTL mapping is generally achieved using bi-parental cross populations (i.e., a cross between two parents which have a contrasting phenotype for the trait of interest are developed). Commonly used populations are recombinant inbred lines (RILs), doubled haploids (DH), back cross, and F₂. An F₂ population is used in the examples herein. However, those of ordinary skill in the art will recognize that other populations may be used.

In an overarching aspect, the present invention provides a tomato plant (Solanum lycopersicum) produced from a recombinational event and having Sw-5, originally introgressed from S. peruvianum, in cis with Ph-3, originally introgressed from S. pimpinellifolium.

### Definitions

Botanical terminology: Linnaeus is considered the father of botanical classification. Although he first categorized the modern tomato as a Solanum, its scientific name for many years has been Lycopersicon esculentum. Similarly, the wild relatives of the modern tomato have been classified within the Lycopersicon genus, like L. pennellii, L. hirsutum, L. peruvianum, L. chilense, L. parviflorum, L. chmielewskii, L. cheesmanii, L. cerasiforme, and L. pimpinellifolium. Over the past few years, there has been movement to reclassify the names of these species. The new scientific name for the modern tomato is Solanum lycopersicum. Similarly, the new names of wild species have been altered: L. pennellii is now Solanum pennellii, L. hirsutum is now S. habrochaites, L. peruvianum is now S. ‘N peruvianumr’ and S. ‘Callejon de Huayles’, S. peruvianum, and S. corneliomuelleri, L. parviflorum is now S. neorickii, L. chmeilewskii is now S. chmielewskii, L. chilense is now S. chilense, L. cheesmaniae is now S. cheesmaniae or S. galapagense, and L. pimpinellifolium is now S. pimpinellifolium [Solanacea Genome Network (2005) Spooner and Knapp; http://www.sgn.cornell.edu/help/about/solanum_nomenclature.html].

Thus, although the names for tomato and its relatives may change, for the purpose of clarification, the modern tomato and its wild relatives are defined herein using the names that fall within the Solanum genus.

As used herein, the term “oomycete” refers to water molds. Oomycetes form a distinct phylogenetic lineage of fungus-like eukaryotic microorganisms. They are filamentous microscopic, absorptive organisms that reproduce both sexually and asexually. Oomycetes occupy both saprophytic and pathogenic lifestyles—and include some of the most notorious pathogens of plants, causing devastating diseases such as late blight. The majority of the plant pathogenic species can be classified into three groups, including the Phytophthora group, which is a genus that includes the species Phytophthora infestans, which causes the disease late blight.

As used herein, the term “allele(s)” means any of one or more alternative forms of a gene at a particular locus, all of which alleles relate to one trait or characteristic at a specific locus. In a diploid cell of an organism, alleles of a given gene are located at a specific location, or locus (loci plural) on a chromosome. One allele is present on each chromosome of the pair of homologous chromosomes. A diploid plant species may comprise a large number of different alleles at a particular locus.

As used herein, the term “locus” (loci plural) means a specific place or places or a site on a chromosome where for example a gene or genetic marker is found. The “Ph locus” refers herein to the location in the tomato genome at which one or more alleles are located, which determine the degree of Phytophthora infestans resistance the plant or plant tissue has. The term “Sw locus” refers herein to the location in the tomato genome at which one or more alleles are located, which determine the degree of TSWV resistance the plant or plant tissue has.

As used herein, the terms “in the coupling phase” and “in cis” refer to a genetic condition in which the alleles of two different loci occur together linked on one (homologous) chromosome. For example, when the alleles Sw-5 and Ph-3 are located on one chromosome homologue, these alleles are “in the coupling phase” or “in cis.” In contrast, if the alleles Sw-5 and Ph-3 are located on different homologous chromosomes of a homologous pair, they are said to be “in the repulsion phase”, or “in trans.”

A “recombinant” or “recombinational event” refers herein to a plant having a new genetic makeup arising as a result of crossing over and independent assortment of the homologous chromosomes.

A “TSWV resistance allele” refers to an allele, which when present in the genome, confers “resistance” to Tomato Spotted Wilt Virus infection and/or damage. A plant or a plurality of plants are said to be “resistant” to TSWV when the plants have a “percentage susceptible” score of between 0% and 10%, or equal to 0% or 10%, using the “TSWV resistance assay” (see below). A plant or a plurality of plants are said to be “susceptible” to TSWV when the plants have a percent susceptible score of above 10%.

A “Phytophthora infestans resistance allele” refers to an allele, which when present in the genome confers resistance to the Phytophthora infestans species. Resistance can come in varying degrees. A plant or a plurality of plants are said to be “highly resistant” to this species when the plants have an average disease score of less than about 1.25, when using the detached-leaf “Phytophthora infestans resistance assay” (see below). For example Ph-3/Ph-3 plants and Ph-3/+ plants are highly resistant. A plant or a plurality of plants are said to have “intermediate” or “moderate” resistance when the plants have an average disease score in the detached leaf assay of about 1.25 or more, but below 2.0. Plants having an average disease score of 2.0 or more using the detached-leaf assay are said to be susceptible. Evaluation in the field is subject to greater variation, thus plants or a plurality of plants are said to have “intermediate” or “moderate” resistance when the plants have an average disease score of about 1.5 or more, but below 2.5. Plants having an average disease score below 1.5 when evaluated in the field are said to have “high resistance,” while plants having an average disease score of 2.5 or more when evaluated in the field are not in these degrees of resistance, but rather are said to be susceptible.

A “TSWV resistance assay” refers to an evaluation of seedlings under greenhouse conditions with inoculation of same with TSWV inoculum and scoring disease symptoms at one or more time points following infection to determine the percentage of plants susceptible to infection (i.e., displaying disease symptoms), as further described in the Example, for a plurality of plants having a specific allelic composition (genotype) at the Sw locus. Briefly, as will be described in the Example, mechanical inoculation of an “HR-1” isolate was performed at least twice, one week apart, with controls included. Plants with stunted growth, necrotic patches, or chlorotic ringspots were considered infected and scored as homozygous susceptible (Sw-5+/Sw-5+). Individual progeny were rated for resistance to TSWV as R (resistant) or S (susceptible) and the percentage of susceptible (S) progenies was calculated. Two separate evaluations were performed in 2008 and 2010 using different recombinant families.

A “Phytophthora infestans resistance assay” refers to a plurality of plants being grown in greenhouse conditions and in the field and testing (1) detached leaves of greenhouse plants, and (2) a field test for the plants in the field themselves, following inoculation with Phytophthora infestans inoculum and scoring the disease symptoms on a scale of 1-5 for detached leaves (after about 7 days) and 0-5 for plants in the field when susceptible checks approached a disease symptom rating of 5, as further described in the Example, for a plurality of plants having a specific allelic composition (genotype) at the Ph locus.

As used herein, the term “heterozygous” means a genetic condition existing when two different alleles reside at a specific locus, but are positioned individually on corresponding pairs of homologous chromosomes in the cell of a diploid organism (e.g., Ph-3/+). Conversely, as used herein, the term “homozygous” means a genetic condition existing when two identical alleles reside at a specific locus, but are positioned individually on corresponding pairs of homologous chromosomes in the cell of a diploid organism (e.g., Ph-3/Ph-3).

As used herein, the term “plant” includes the whole plant or any parts or derivatives thereof, such as plant cells, plant protoplasts, plant cell tissue cultures from which tomato plants can be regenerated, plant calli, plant cell clumps, and plant cells that are intact in plants, or parts of plants, such as embryos, pollen, ovules, fruit (e.g., harvested tomatoes), flowers, leaves, seeds, roots, root tips and the like.

A “molecular assay” (or test) refers to a (DNA based) assay that indicates (directly or indirectly) the presence or absence of a particular allele at the Sw or Ph locus. In addition it allows one to determine whether a particular allele is homozygous or heterozygous at the Sw or Ph locus in any individual plant. For example, in one embodiment a nucleic acid linked to the Sw or the Ph locus is amplified using PCR primers, the amplification product is digested enzymatically and, based on the electrophoretically resolved patterns of the amplification product, one can determine which Sw or Ph alleles are present in any individual plant and the zygosity of the allele at the Sw or Ph locus (i.e., the genotype at each locus). Marker systems are based on detection of fragment length polymorphism due to differences in size [examples are simple sequence repeat (SSR) and Insertion/deletion (INDEL) polymorphisms]; fragment size polymorphisms due to indirect detection of a single nucleotide polymorphism (SNP) change due to restriction enzyme digestion [examples are cleaved amplified polymorphic sequence (CAPS), sequenced characterized amplified region (SCAR)]; and differences due to SNPs which are assayed such as allele-specific primer extension (ASPE), ligation based assays, and similar assays.

As used herein, the term “variety” or “cultivar” means a plant grouping within a single botanical taxon of the lowest known rank, which can be defined by the expression of the characteristics resulting from a given genotype or combination of genotypes.

As used herein, the term “wild type”, means the naturally occurring allele found within S. lycopersicum. At the Phytophthora resistance locus Ph, and the TSWV locus Sw, these wild type alleles from S. lycopersicum confer susceptibility to these pathogens and are designated as Ph+ and Sw+ herein, or simply “+”.

As used herein, the term “variant” or “polymorphic variant” refers to nucleic acid sequences that are essentially similar to a given nucleic acid sequence. For example, the term “variants thereof” or “variants of any of SEQ. ID. NOS. 1-10” refers to a polynucleotide sequence having one or more (e.g., two, three, four, five or more) nucleotides deleted (deletion variants) from said polynucleotide sequence or having one or more nucleotides substituted (substitution variants) with other nucleotides or one or more nucleotides inserted into said polynucleotide sequence (insertion variants).

Variants of SEQ. ID. NOS. 1-10 include any nucleotide sequences that are “essentially similar” to any of SEQ. ID. NOS. 1-10. Sequences which are essentially similar to SEQ. ID. NOS. 1-10 are nucleic acid sequences comprising at least about 90%, more preferably 91%, 92%, 93%, 94%, 95%, 96%, 97%, 98% or 99% or more nucleic acid sequence identity to one or more sequences of SEQ. ID. NOS. 1-10, when optimally aligned using, for example, the Needleman and Wunsch algorithm, with, for example, the programs GAP or BESTFIT using default parameters. GAP default parameters are a gap creation penalty=50 (nucleotides) and gap extension penalty=3 (nucleotides). For nucleotides the default scoring matrix used is nwsgapdna [Henikoff & Henikoff, 1992, PNAS 89, 915-919]. Sequence alignments and scores for percentage sequence identity may be determined using computer programs, such as the GCG Wisconsin Package, Version 10.3, available from Accelrys Inc., 9685 Scranton Road, San Diego, Calif. 92121-3752, USA or the open-source software Emboss for Windows (version 2.7.1-07). Variants also include fragments or parts of any of these sequences. Another example of a commonly used program for sequence alignment is the Basic Local Alignment Search Tool (BLAST) [Altschul, Stephen F., Thomas L. Madden, Alejandro A. Schaffer, Jinghui Zhang, Zheng Zhang, Webb Miller, and David J. Lipman (1997),n “Gapped BLAST and PSI-BLAST: a new generation of protein database search programs”, Nucleic Acids Res. 25:3389-3402 ]. BLAST finds regions of local similarity between sequences. The program compares nucleotide or protein sequences to sequence databases and calculates significance of matches. BLAST can be used to infer functional and evolutionary relationships as well as help identify members of gene families (See the National Center for Biotechnology Information or its webpages at: and http://www.ncbi.nlm.nih.gov/).

### Plants and Plant Parts According to the Invention

In one embodiment, the present invention provides a tomato plant (Solanum lycopersicum) comprising in its genome the Sw-5 allele, originally introgressed from S. peruvianum, in cis with the Ph-3 allele, originally introgressed from S. pimpinellifolium, as well as plant cells and tissues, seeds or fruit of such plants. These plants can be made, for example, by crossing publicly available commercial varieties, each comprising a (preferably fixed) allele of interest (here Sw-5 or Ph-3) and by selecting recombinant plants, comprising Sw-5 or Ph-3 in cis, from the F₂ plants obtained from the cross, or from any further generation obtained by further selling or crossing of the F₁ (e.g., an F₂ or backcross population). As the incidence of recombination is exceedingly low, requiring a large numbers of progeny to be screened, the selection is preferably carried out using one or more Sw allele specific or allele discriminating molecular assays, such as CAPS and SCAR, as described in the Example. More specifically, a CAPS assay was used to detect a single nucleotide polymorphism (SNP) in the Example to select for Ph-3. In the CAPS assay, 2 primer pairs are used in a PCR reaction, followed by an enzyme restriction and detection of the fragments obtained to detect polymorphisms between the PCR amplification products. And, in the Example, a SCAR assay was used to select for Sw-5. Again, the use of these particular assays is exemplary and other assays, such as allele-specific primer extension (ASPE) or GoldenGate (ligation based), which are known to those of ordinary skill in the art, may be used.

It is understood that routine experimentation can be used to develop a similar assay. For example “variants” of any of the primer sequences may be used, or primers or probes which hybridize to other parts of the genome near or on the Sw and Ph locus.

Plants comprising Sw-5 and Ph-3 in cis may be homozygous or heterozygous. These plants may be used in further crosses to transfer the alleles as a single unit to other tomato plants to generate, for example, hybrids or inbreds. In one exemplary embodiment, hybrid plants are provided that comprise the Sw-5 and Ph-3 alleles in coupling phase and susceptible, or Sw+ and Ph+, alleles on the homologous chromosome. These plants have the benefit of having significantly reduced or no genetic drag symptoms normally associated with the Sw-5 and Ph-3 alleles when these alleles are present in the homozygous condition.

“Genetic drag symptoms” refer to one or more symptoms selected from the group of auto-necrosis, longer internodes, smaller fruits, less fruit set and horticulturally inferior architecture, compared to a plant lacking the Sw-5 and Ph-3 alleles. Those skilled in the art will recognize that such symptoms of genetic drag will adversely affect the commercial acceptance of inbred or hybrid plant lines by growers. Generally, the presence of an adverse level of genetic drag can be determined by the presence of one or more of these symptoms to such a degree that the plant line becomes commercially unacceptable.

The tomato plants of this invention comprise an average Phytophthora infestans resistance score of less than about 1.25 on a 1-5 scale with 1 being no lesions and 5 being completely covered with lesions or dead as determined by the described detached leaf assay; and/or an average Phytophthora infestans resistance score of less than about 1.5 on a 0-5 scale with 0 being no lesions and 5 being completely covered with lesions or dead as determined by the field assay. In addition, these plants are resistant to TSWV and have an average TSWV resistance score of lower than or equal to 10% susceptible, as determined in the described assay.

### Plants Comprising Other Phytophthora and TSWV Resistance Alleles

In another embodiment, the invention provides a method of making and/or selecting the above recombinant, as well as a method for making and/or selecting other recombinants having at least one Sw resistance allele and at least one Ph resistance allele in coupling phase (in cis), preferably on chromosome 9 of S. lycopersicum. Such plants are characterized by having high resistance, or intermediate or moderate resistance, to Phytophthora infestans, and by having resistance or intermediate resistance to TSWV, when using the resistance assays described herein. Also provided are recombination events made using this method, as well as tissues, cells, seeds and fruit of these plants and the use of any of these plants to generate hybrid or inbred plants comprising the Sw and Ph resistance alleles in cis.

In one embodiment, the present invention relates to the making of a tomato plant that comprises an allele that confers resistance to TSWV in the coupling phase with an allele that confers resistance to Phytophthora infestans. The allele that confers resistance to TSWV and the allele that encodes for resistance to Phytophthora infestans may originally derive from different germplasm sources (i.e., different species of tomato), such as, but not limited to, S. lycopersicum, S. pimpinellifolium, S. cheesmanii, S. neorickii, S. chmielewskii, S. habrochaites, S. pennellii, S. peruvianum, S. chilense, or S. lycopersicoides.

Thus, in one embodiment a method for making a S. lycopersicum plant comprising at least one TSWV resistance allele and at least one Phytophthora infestans resistance allele in the coupling phase at two loci is provided, wherein the method comprises the steps of: (a) crossing a Solanum plant comprising a TSWV resistance allele with a Solanum plant comprising a Phytophthora infestans resistance allele, (b) analyzing progeny of the cross for the presence of the resistance alleles at each of the two loci using one or more molecular assays, and (c) selecting one or more plants comprising the resistance alleles in the coupling phase.

Resistance assays may be optionally performed at any stage of the method. A further optional step (d) comprises selfing the plant obtained or crossing the plant obtained with another tomato plant to create a hybrid plant. In one embodiment the plant obtained by the method is resistant to TSVW and highly resistant to oomycetes (as defined).

The starting plants of step (a) can be selected using pathological tests. They may be wild or cultivated plants, or modified plants, such as mutagenized or transformed plants. For example, approaches such as TILLING [Targeting Induced Local Lesions IN Genomics; McCallum et al., 2000, Nat Biotech 18:455, and McCallum et al. 2000, Plant Physiol. 123, 439-442] or ECOTILLING [Henikoff et al 2004, Plant Physiology Preview May 21, 2004] may be used to generate and/or select plants with modified pathogen resistance and/or mutations in alleles of the Sw or Ph locus. These plants may then be used as sources of Sw and Ph resistance alleles.

The progeny of the cross are then analyzed, using one or more molecular assays according to the invention (described below). The progeny analyzed and from which plants are selected may be any of various generations of progeny, such as the F₂, F₃ generation, etc., a backcross generation (BC₁, BC₂, etc) etc., depending on the crossing/selection scheme desired and the alleles present in the plants used. The molecular assay described herein is carried out on F₂ plants (and subsequent generations), but this is not limiting. Also, progeny of different generations may be repeatedly tested using pathological assays and/or one or more molecular assays. Several molecular assays may be carried out in one generation, or one or more different assays may be carried out in different generations. Thus, steps (a), (b) and/or (c) may be repeated several times. The aim is to identify recombinants comprising the desired Sw and Ph resistance alleles in the coupling phase (step c). In this method any Sw resistance allele may be combined (in the coupling phase) with any Ph resistance allele.

The plants can be distinguished from other plants using molecular assays, based on, for example a nucleic acid sequence near or at the Sw locus and near or at the Ph locus. These analyses allow the allelic make up at these two loci to be determined. For example, one or more PCR based assays as described elsewhere herein are used to distinguish between different genotypes at the Sw and Ph loci and to select a recombinant plant having the desired alleles in coupling phase.

The selection of a recombinant plant comprising a Sw and a Ph resistance allele in the coupling phase and the introgression of this single Mendelian unit into other plants may be achieved using a combination of molecular biology, plant pathology and traditional breeding techniques. In a preferred approach, the present invention uses molecular biology techniques to discriminate between different alleles at the Sw and Ph loci to combine the desirable alleles for TSWV resistance and Phytophthora infestans resistance into the genome of cultivated tomato in cis. The present invention facilitates the breeding of tomato hybrids with multiple resistance to both TSWV and Phytophthora infestans while enhancing the plant breeder's ability to mask the genetic drag that is typically associated with these traits, while retaining the freedom to combine the Sw and Ph resistance with other resistance genes, quantitative trait loci conferring resistance, or other yet to be discovered resistance genes present in this gene cluster. Such other genes that may be combined together with the tomato having Sw-5 and Ph-3 in coupling phase may include one or more of Bw1, Bw3, Bw4, Bw5, multiple QTL (Cm1.1-Cm10.1), Cor-1, Cor-2, Rcm 2.0, Rcm 5.1, Pto, Prf, Pto-2, Rx-1, Rx-2, Rx-3, Rx-4, Xv-3, FrI, Asc, Ad, multiple QTL (no designations), Cf-1, Cf-2, Cf-4, Cf-5, Cf-6, Cf-7, Cf-9, Cf-10, Cf-12, Cf-19, I, I2, I3, Lv, OI-1, OI-3, py-1, Sm, Se, Ve, Hero, Tv-1, Tv-2, Mi, Mi-3, Mi-4, Ph-1, Ph-2, Ph-3, Ph-4, Ph-5, Phf, Phf-2, Ora, pot-1, Sw-5, Sw-7, Tm1, Tm2, Tm2a, Ty-1, Ty-2, Ty-3, Ty-4, rt, Am, and Cmr. Further, genes that may be coupled with the tomato having Sw-5 and Ph-3 in coupling phase may be one or more genes that provide resistance to one or more of Bacterial Wilt, Bacterial Canker, Bacterial Speck, Bacterial Spot, Fusarium crown rot, Alternaria Stem Canker, Alternaria collar rot, Early Blight, Leaf Mold, Fusarium wilt, Powdery Mildew, Corky Root, Stemphylium Leaf Blight, Septoria Leaf Spot, Verticillium Wilt, Potato Cyst Nematode, Greenhouse Whitefly, Root Knot Nematode, Late Blight, Broomrape, and Alfalfa Mosaic Virus. Those skilled in the art will recognize that the above lists of genes and diseases are not necessarily comprehensive, and other genes may be provided.

By way of example, but not of limitation, the present invention provides for the development of tomato germplasm comprising any Sw resistance allele and any Ph resistance allele in the coupling phase, preferably as an in cis co-inherited unit located on chromosome 9. Once plants have been identified that have high levels of resistance, (e.g., comparable to resistance levels provided by Sw-5 and Ph-3), the nucleic acid regions disclosed herein, which are closely linked to the Sw and Ph loci, can be sequenced and the sequence information (of the linked marker region) used to develop a molecular assay for the alleles found in those plants. Alternative methods exist for identifying Sw or Ph resistance alleles in various germplasm, as will be apparent to those of skill in the art. Further details of such methods are provided below.

As mentioned previously, the present invention uses a combination of molecular biology, plant pathology and traditional breeding techniques. In one embodiment, the molecular biology techniques used involve marker assays that employ, for example, nucleic acid primers which hybridize (and amplify) a nucleic acid region linked to the Sw and/or Ph locus, which will be discussed in more detail below. The present invention not only contemplates the specific assays disclosed in the Example, which involve the Sw-5 and Ph-3 alleles, but any assays that can be developed and used to introgress into tomato any allele that encodes for resistance to TSWV in the coupling phase with any allele that encodes for resistance to Phytophthora infestans. For example, the present invention contemplates introgressing into tomato any variants (for example orthologs or evolutionarily diverged natural alleles or alleles generated by mutagenesis) of the Sw and/or Ph loci and the generation of plants comprising the alleles in cis.

Also provided herein are plants obtainable by any of the methods described and the use of those plants as parent in a cross with another S. lycopersicum plant. It should be noted that the present invention is in no way technically limited to one or more specific varieties of tomato, but is generally applicable to tomato plants (including inbreds, hybrids, etc.).

### Molecular Assays According to the Invention

A number of molecular assays are provided herein which discriminate between the presence or absence of Sw-5 and Sw+ at the Sw locus and between the presence or absence of Ph-3 and Ph+ at the Ph locus of a plant. One or more of these assays can be used in marker-assisted selection (i.e., to determine the allelic make up of plants at the Ph and Sw locus and to select plants having the desired Sw and Ph resistance alleles in coupling phase). Similar assays can be developed for any Sw and Ph resistance alleles, using routine molecular biology techniques. For example, any fragment of 10, 12, 14, 16, 18, 20, 21, or more consecutive nucleotides of SEQ. ID. NOS. 1-10 (or variant nucleic acid sequences) may be used to design PCR primer pairs or probes for nucleic acid hybridization and to develop discriminating molecular assays based on the nucleic acid information of the region amplified by such primer pairs or of the nucleic acid sequence to which such probes hybridize. The exact type of assay developed is not important, as long as it can discriminate between Sw resistance alleles and Ph resistance alleles and homozygosity/heterozygosity at the Sw and/or Ph locus. Examples of various types of assays are given below and in the Example.

In order to perform the marker-assisted selection in the methods of the present invention, the subject tomato plants or plant parts are, for example, first subjected to DNA extraction, the techniques of which are known in the art [See, for example, Hnetkovsky et al., Crop Sci., 36(2): 393-400 (1996), incorporated by reference herein in its entirety]. Once the extraction is complete, a molecular assay can be performed, including, but not limited to, a cleaved amplified polymorphic sequence (CAPS) assay [see Akopyanz et al., Nucleic Acid Research, 20:6221-6225 (1992) and Konieczny & Ausubel, The Plant Journal, 4:403-410 (1993), incorporated by reference herein in their entireties] or a sequence characterized amplified region (SCAR) assay.

The cleaved amplified polymorphic sequence (CAPS) method is a technique in molecular biology for the analysis of genetic markers. It is an extension to the restriction fragment length polymorphism (RFLP) method, using polymerase chain reaction (PCR) to more quickly analyze the results. Basically, CAPS polymorphisms are differences in restriction fragment lengths caused by single nucleotide polymorphisms (SNPs) or insertion/deletion markers (INDELs) that create or abolish restriction endonuclease recognition sites in PCR amplicons produced by locus-specific oligonucleotide primers. CAPS works on the principle that these genetic differences create or abolish restriction endonuclease restriction sites, and thus these differences can be detected in the resulting DNA fragment length after digestion. And so, PCR amplification is directed across the altered restriction site, and the products digested with the restriction enzyme. When fractionated by agarose or acrylamide gel electrophoresis, the digested PCR products will give readily distinguishable patterns of bands, thereby allowing one to identify the alleles present.

A SCAR assay involves amplifying DNA at the locus (e.g., a specific locus near the Sw locus or the Ph locus) by PCR followed by digestion with restriction enzymes. Polymorphisms between the nucleic acid sequences differentiates between different alleles (such as, but not limited to, the Ph+ and Ph-3 alleles) by resulting, for example, in different sized restriction fragments. SCAR analysis provides this by revealing specific nucleic acid polymorphisms within the tested DNA. Because a tomato line having the Sw-5 allele does not share identical DNA sequences with a line having Sw+, they can be distinguished based on the various nucleic acid polymorphisms. These nucleic acid regions that comprise polymorphisms and can be used to make the identification are referred to as SCAR regions (i.e., regions of known sequence, varying in sequence between species, which can be used to identify or distinguish between a related group of organisms when amplified by PCR or similar techniques).

Further, as is known to those of ordinary skill in the art, PCR is a technique to amplify a single or few copies of a particular nucleic acid sequence, e.g., DNA (the target DNA sequence), across several orders of magnitude, thereby generating thousands to millions of copies of the sequence. PCR relies on “thermal cycling,” which includes cycles of repeated heating and cooling of the DNA and other reaction components to cause DNA denaturation (i.e., separation of the double-stranded DNA into its sense and antisense strands) followed by enzymatic replication of the DNA. The other reaction components include short oligonucleotide DNA fragments known as “primers,” which contain sequences complementary to at least a portion of the target DNA sequence, and a DNA polymerase. These are components that facilitate selective and repeated amplification of the target sequence. As PCR progresses, the DNA generated is itself used as a template for further replication in subsequent cycles, creating a chain reaction in which the target DNA sequence is exponentially amplified. The DNA polymerase used in PCR is thermostable (and thus avoids enzyme denaturation at high temperatures) and amplifies target DNA by in vitro enzymatic replication. One such thermostable DNA polymerase is Taq polymerase, an enzyme originally isolated from the bacterium Thermus aquaticus. The DNA polymerase enzymatically assembles a new DNA strand from deoxynucleoside triphosphates (dNTPs) by using the denatured single-stranded DNA as a template. The initiation of DNA synthesis and the selectivity of PCR results from the use of primers that are complementary to the DNA region targeted for amplification under specific thermal cycling conditions.

Nucleic acid primers and enzymes are employed in these assays in order to identify which alleles are present at the Sw and/or Ph loci in the genome of a tomato plant, and, if said alleles are present, whether the alleles are present in a homozygous or heterozygous condition. The information obtained from both loci is used to identify those plants that have specific allelic combinations at the Sw and Ph loci in the coupling phase (i.e., in cis). The information may also be used to map the location of the alleles in general and/or relative to one another.

To create an MAS test, one skilled in the art begins by comparing the DNA sequence from the donor source (i.e., the germplasm containing the disease resistance trait) with the corresponding DNA sequence from the recipient source (i.e., the germplasm containing the susceptible ‘+’ alleles for the specific pathogen). Alternatively, a sequence comparison between DNAs from the donor and recipient can be performed at corresponding positions in the genome that are tightly linked genetically to the trait of interest. For the Sw and Ph loci, identification of polymorphisms near these traits are known in the art. As is known to those of ordinary skill in the art, sequence comparison can be direct (e.g., based on sequencing reaction) or indirect (e.g., based on digestion of PCR primers with randomly chosen restriction endonucleases).

It is understood that the herein described CAPS and SCAR assays can be easily modified or replaced by other molecular assays and can easily be developed for any allele of the Sw− and/or Ph− locus. Also, the assay may be based on other polymorphisms than SNPs, such as deletion, insertion or substitution of two or more nucleotides.

As already mentioned, specific or degenerate primers can be designed which hybridize to and amplify all or part of markers TG328, TG591, SCAR421, or CT202, or all or part of any variants thereof or flanking regions in the genome. Alternatively, the primers may be designed to amplify parts of the resistance alleles directly or other nucleic acid regions near the Sw− and Ph loci. Moreover, when desired, the primers of the present invention can be modified for use in other marker-assisted selection assays, such as, but not limited to, the TAQMAN™ assay from Applied Biosystems, Foster City, Calif., using techniques known in the art, including, but not limited to, those described in U.S. Pat. Nos. 5,464,746, 5,424,414 and 4,948,882 (each of which is incorporated by reference herein in its entirety).

To design a new molecular test to distinguish a new Sw or Ph resistance allele, those skilled in the art recognize that one would, for example, first determine the DNA sequence at the marker locus on or near the Sw or Ph locus (using e.g., PCR amplification and the primer pairs described herein and sequencing of the amplification product), and then compare the sequence with the corresponding DNA of the other marker sequences (on or near other alleles of the Sw and Ph locus). With this DNA comparison, those skilled in the art could identify either new sequence polymorphisms or whether existing polymorphisms previously uncovered with other comparisons remain. Using these data, one can either design a new molecular test, or use an existing test to facilitate the selection of any allelic combination at the Sw and Ph loci together in cis.

The above-described assays can be used individually and collectively in a breeding program to facilitate the breeding and/or selection of tomato plants that contain the Sw resistance allele and the Ph resistance allele in the coupling phase.

One non-limiting example of how these methods can be used is described below, for the selection of a plant comprising Sw-5 and Ph-3 in coupling phase. A first inbred tomato line may be crossed with a second inbred tomato line to produce a hybrid plant. One tomato plant used in the cross contained the Sw-5 allele and the second plant the Ph-3 allele in its genome. A resulting plant (F₁ hybrid) is then allowed to self-pollinate, fertilize and set seed (F₂ seed). The F₂ plants are grown from the F₂ seed (or further selfed or crossed, e.g., backcrossed to one of the parents). These plants are then subjected to DNA extraction, the techniques of which are known in the art [See Hnetkovsky et al., 1996, Crop Sci., 36 (2): 393-400, incorporated by reference herein in its entirety] and PCR is carried out directly on crushed tissue samples.

Thus, the above-described assays can be used to identify F₂ plants (or other progeny) that contain the Sw-5 allele in a heterozygous state and the Ph-3 allele in a homozygous state. Alternatively, one can identify an F₂ plant that contains the Sw-5 allele in a homozygous state and the Ph-3 allele in the heterozygous state. Thus, using one or more molecular assays, such as the SCAR assays described, recombinant plants can be identified which comprise Sw-5 and Ph-3 in coupling.

Because recombination between the Sw and Ph loci is low, finding recombinants in the F₂ generation is rare. The assays described herein can also be used to determine if the Sw-5 and/or Ph-3 alleles are present in the genome of the plant in the homozygous or heterozygous condition. Depending upon the results of the assay(s), further breeding and molecular characterization may be necessary. For example, if the goal of the breeding program is to create an inbred line and the results of one or more of the above-described assays for a specific tomato plant being tested reveal that the plant contains the Ph-3 allele in its genome in a homozygous condition and the Sw-5 allele in a heterozygous condition, then that plant may be subjected to further self-fertilization, breeding and/or molecular characterization using one or more of the assays described herein, until it has been determined that said plant and its progeny, after selfing, contains both the Ph-3 allele and the Sw-5 allele in its genome under homozygous conditions. Once the Ph-3 and Sw-5 alleles are created in the coupling phase, or in cis, they will be inherited together. This heritable block of multiple resistance alleles provides the plant breeder with flexibility in creating new hybrids, while also allowing the plant breeder the ability to mask the genetic drag effects of the wild species introgressions with the second inbred parent. Also, easy combination with other resistance genes, such as those described above, is possible.

As mentioned briefly above, the methods of the present invention can be used to create new and superior inbred lines. These inbred lines can be used in subsequent breeding to create hybrid tomato plants that are resistant to TSWV and Phytophthora infestans and also possess other commercially desirable characteristics. Such inbred lines are useful in breeding because these lines allow for the transfer of the Sw-5 and Ph-3 alleles as a single co-inheritable unit that facilitates rapid breeding. Moreover, the above-described methods are also useful in confirming that an inbred line does in fact contain the Sw-5 allele and the Ph-3 allele in its genome in a homozygous condition and is maintaining its homozygosity. Once this confirmation is obtained, the inbred line can be used in crosses with a second inbred line to transfer the Sw-5 allele and Ph-3 allele to a hybrid tomato plant as a single co-inheritable unit. The second inbred line can carry the wild type alleles Sw+ and Ph+ to mask the effects of genetic drag.

### Kits According to the Invention

In yet a further embodiment, molecular assays for determining the allelic composition at the Sw and/or Ph locus are provided. Such assays involve extracting DNA from one or more tomato plants, amplifying part of the DNA linked to or on the Sw and/or Ph locus using at least one PCR primer pair, optionally restricting the amplification product with one or more restriction enzymes, and visualizing the DNA fragments.

Further provided is a detection kit for determining the allelic make up of a plant or plant tissue at the Sw locus and at the Ph locus. Such a kit comprises one or more primer pairs, such as SEQ. ID. NOS. 5 and 6 and/or SEQ. ID. NOS. 7 and 8 and/or SEQ. ID. NOS. 9 and 10, or variants thereof. Further, instructions and optionally plant material or DNA (e.g., of control tissue) may be included.

Thus, multiple tomato breeding and hybrid lines having at least one TSWV resistance allele and at least one Phytophthora infestans resistance allele are described herein. Furthermore, TSWV and Phytophthora infestans resistance alleles, as well as resistance assays are described herein, and such alleles in the coupling phase in the tomato genome are described herein. Moreover, numerous Solanum lycopersicum plants for use in the production of the hybrid Solanum lycopersicum plants are known in the art.

Additionally, guidance to produce the claimed Solanum lycopersicum plant is described herein. This specification enables multiple breeding lines and hybrid lines comprising within their genome at least one TSWV resistance allele and at least one Phytophthora infestans resistance allele. This specification also provides pathogen assays for identifying resistance in tomato plants, and molecular marker tests for identifying Sw and Ph genotypes for use in breeding methods and distinguishing between various alleles. Moreover, this specification provides herein breeding methods for combining Sw and Ph alleles. Those of ordinary skill in the art thus are guided on the conditions and approaches that can be used to identify, confirm, and introduce into other Solanum lycopersicum lines, TSWV and Phytophthora infestans resistance alleles in the coupling phase.

Further, the Sw and Ph alleles (such as Sw-5 and Ph-3) reside on an area of chromosome 9 known to have severely suppressed recombination, and up until the present invention, there has remained a need to observe a recombination event in this area. Because the alleles are tightly linked, a skilled artisan would not have expected to obtain plants having recombination between those loci. Thus, when breeding from parents where one has the desired allele at the Sw locus and the other has the desired allele at the Ph locus, one would not anticipate being successful in obtaining a plant with both alleles. However, the methods described in the present application do allow for both alleles to be obtained in a plant in coupling phase.

Further, as described previously, prior art patents have been cited and theoretically a breeder could generate billions of different genetic combinations via crossing, selfing, and selection; however, a breeder has no direct control at the cellular level and therefore, two breeders will never develop the same line or even very similar lines having precisely the same traits. However, these comments must be tempered by the fact that a skilled artisan, in light of the present disclosure, would understand that different lines not having precisely the same traits being referred to in such a previous patent can arise due to differences in the amount of DNA flanking an allele (or alleles) that is introgressed into progeny of a cross, and perhaps other variations associated with phenotypic characteristics that are not being actively selected by a breeder. For example, while plants may not be exactly the same at the cellular level, the plants may be indistinguishable at the macroscopic phenotypic level depending on which traits are examined. Given the current disclosure, once a trait or coupled traits of interest are fixed in a population, in this case the Sw and Ph alleles, they can be transmitted and bred into other members of the species, and variations at loci not related to the alleles of interest can be minimized by back crossing to a recurrent parent.

The various aspects of the present invention will be described in greater detail with respect to the following Example.

### EXAMPLE

### Background

Starting with two parental lines, each containing a fixed allele of interest that are closely linked, those skilled in the art will recognize that there are several genetic strategies possible to achieve the goal of combining these traits of interest in cis. All of these strategies, however, begin by crossing the parental lines, each containing a trait of interest to make an F₁ hybrid. The F₁ plant can either be self pollinated to create a segregating F₂ population, or it can be backcrossed to either parental line. Irrespective of the crossing strategy, those skilled in the art will recognize that novel recombinants of interest can be created as the F₁ plant produces gametes through the process of meiosis.

In this Example, though by no means limiting, an F₂ strategy was followed to combine the Sw-5 and Ph-3 alleles in cis. Specifically, a cross between inbred breeding lines NC 946-1(2004)-11 (designated as NC946) and NC 0592-8-10 (designated as NC592) was made in North Carolina and the F1 was self pollinated to provide the F₂ population. NC 0592-8-10 is a Ph-3/Ph-3 inbred line and was derived from NC 0483×NC 25P (Gardner and Panthee, 2010. ‘Plum Regal’ Fresh market plum tomato hybrid and its parents, NC 25P and NC 30P. HortScience, 45: 824-825) whereas NC 946-1(2004)-11 is a Sw-5/Sw-5 inbred line derived from NC EBR-7×‘Amelia’. 1152 F₂ seedlings were sampled for DNA extraction, and the genotype at the Sw locus and the Ph locus was determined for each sample based on linked DNA-based genetic markers using methods described herein. Recombinants were expected to be rare, as it has been well documented that recombination is suppressed in this region of chromosome 9. Eight such recombinants were discovered. The Sw-5 and Ph-3 introgessions are known to cause genetic drag separately. Therefore, because each of the 8 recombinants discovered likely are unique recombinational events, this was an opportunity to reduce drag associated while introgressing these traits.

In 2008, the eight F₂ plants were self-pollinated to create F₃ families, and were sown in the field in Wooster, Ohio in June 2008. Using methods described herein, the Ph-3 and Sw-5 alleles were fixed in the homozygous condition.

The genetic distance between the Ph and Sw loci determines the relative frequency that the recombinant gametes will be produced. Because the Ph and Sw loci are closely positioned and recombination is suppressed in this region of chromosome 9, those skilled in the art would expect that the number of recombinant gametes would be very low compared with gametes having the parental arrangement of genes.

A series of molecular tests was then performed (the F₃ plants were genotyped by molecular markers), described herein, to identify recombinants having the Ph-3 and Sw-5 alleles in cis (i.e., individuals homozygous for recombination events), because identifying plants with in cis pairing through phenotypic pathology screening was not possible without multi-year, multi-generational screening of large numbers of plants. Those skilled in the art will recognize that the phenotypic identification of this in cis pairing is possible, but it will be apparent that the molecular identification method described herein is a faster and much more efficient method to identify this useful combination of alleles.

To ensure that the molecular testing accurately predicted the resistance phenotype, these fixed lines were tested for Phytophthora infestans resistance according to methodology described herein. Table 1 (below) shows that certain of these lines, having the unique in cis arrangement of the Ph-3 and Sw-5 alleles, were highly resistant to Phytophthora infestans.

These same lines were also tested for TSWV using methodology described herein. Certain of these lines, having the unique in cis arrangement of the Ph-3 and Sw-5 alleles, were resistant to TSWV. And thus, referring to Table 1, certain lines and their progeny were created that exhibit resistance to both TSWV and Phytophthora infestans.

This combination of the Ph-3 and Sw-5 efficacious resistance alleles in cis allows tomato breeders to create tomato hybrids that are resistant to TSWV and highly resistant to Phytophthora infestans, while retaining the freedom of having a second inbred parent to either mask the genetic drag, or delivering additional resistance genes, or yet to be discovered resistance alleles in this disease cluster. This novel approach provides the tomato grower the opportunity to control a number of disease pathogens, with acceptable horticultural qualities without relying exclusively on chemical pesticides for control, or relying on transgenic resistance strategies.

### Materials and Methods

Plant material. As described above, the F₂ population, segregating for Ph-3 and Sw-5, was derived from NC 946×NC 592 and was utilized to identify coupling phase Ph-3/Sw-5 recombinants. Putative F₂ plants in coupling phase for Ph-3 and Sw-5 were selected and self-pollinated to create F₃ families. These families were sown in the field in Wooster, Ohio, in 2008 and genotyped by molecular markers to identify individuals homozygous for recombination events. Presumed Sw-5/Ph-3 homozygous plants were visually inspected for fruit size, shape, and number to identify selections with desirable horticultural characteristics. For each original F₂ recombinant, at least three randomly selected F_{3:4} families with at least seven individuals per family were evaluated for resistance to late blight and TSWV. The results of this study are shown in Table 1, below, and will be referred to through the remainder of this Example.

TABLE 1
Genotype and phenotype of F₂ individuals selected as coupling phase recombinants for Ph-3 and Sw-5 genes in tomato.

| | | | | | | Resistance rating to | | Percent susceptible | |
|---|---|---|---|---|---|---|---|---|---|
| | Markers | | | | | late blight (Ph-3)^{v} | | to TSWV (Sw-5)^{u} | |
| F₂ recombinants^{z} | TG328^{y} | TG591^{y} | SCAR421^{x} | F_{3:4} family^{w} | Generation | Detached leaf | Field | 2008 | 2010 |
| 08-5401 | H^{t} | H | T^{s} | 08-5401-04 | F₄ | 1.2 | | 0 | |
| | | | | 08-5401-04 | F₅ | | | | 0 |
| | | | | 08-5401-25 | F₄ | | 3.9 | | |
| | | | | 08-5401-31 | F₄ | 1.3 | | 0 | |
| 08-5402 | H | H | T | 08-5402-03 | F₄ | 1 | | 57.1 | |
| | | | | 08-5402-20 | F₄ | | 0.6 | | 46.1 |
| | | | | 08-5402-22 | F₄ | 1 | | 22.2 | |
| | | | | 08-5402-31 | F₄ | | 1.3 | | 66.7 |
| 08-5403 | P^{r} | P | H | 08-5403-05 | F₄ | 1 | | 0 | |
| | | | | 08-5403-07 | F₄ | 1.3 | | 7.1 | |
| | | | | 08-5403-07 | F₅ | | | | 0 |
| | | | | 08-5403-13 | F₄ | | 1 | | 0 |
| | | | | 08-5403-25 | F₄ | | 0.9 | | 0 |
| 08-5404 | H | T | T | 08-5404-01 | F₄ | 1.3 | | 9.1 | |
| | | | | 08-5404-14 | F₄ | 2.2 | | 6.7 | |
| | | | | 08-5404-15 | F₄ | | 3.9 | | 0 |
| | | | | 08-5404-38 | F₄ | | 2.4 | | 0 |
| 08-5405 | H | H | T | 08-5405-01 | F₄ | | 2.5 | | 0 |
| | | | | 08-5405-09 | F₄ | 1.5 | | 0 | |
| | | | | 08-5405-11 | F₄ | 1 | | 0 | |
| | | | | 08-5405-18 | F₄ | | 1.1 | | 0 |
| 08-5406 | H | T | T | 08-5406-20 | F₄ | 1 | 3 | 0 | |
| | | | | 08-5406-22 | F₄ | 2.7 | | 0 | 0 |
| | | | | 08-5406-27 | F₄ | | 0 | | |
| 08-5407 | P | P | H | 08-5407-05 | F₄ | 1 | | 100 | |
| | | | | 08-5407-17 | F₄ | 1 | | 100 | |
| | | | | 08-5407-19 | F₄ | | 0.3 | | 100 |
| | | | | 08-5407-26 | F₄ | | 0.5 | | 100 |
| 08-5408 | H | T | T | 08-5408-01 | F₄ | | 0.8 | | 5.6 |
| | | | | 08-5408-07 | F₄ | 1.2 | | 0 | |
| | | | | 08-5408-07 | F₅ | | | | 0 |
| | | | | 08-5408-09 | F₄ | | 0.9 | | 0 |
| | | | | 08-5408-16 | F₄ | 1 | | 0 | |
| Resistant control^{q} | | | | | | 1 | 0 | 10 | 0 |
| Susceptible control^{p} | | | | | | 4.4 | 5 | 100 | 100 |
| Mean | | | | | | 1.5 | 1.7 | 22.9 | 19.9 |
| LSD_{(0.05)} | | | | | | 2 | 1.3 | 20.6 | 15 |
| P value^{o} | | | | | | <0.0001 | <0.0001 | <0.0001 | <0.0001 |

^{z}Individuals derived from a cross between resistant parents NC592 and NC946 were selected based on molecular markers.
^{y}Markers linked with Phytophthora infestans resistance gene Ph-3.
^{x}Marker linked with TSWV resistance gene Sw-5.
^{w}Generated from F₂ recombinants by selecting for individuals homozygous for Ph-3 and Sw-5. Randomly selected progenies were then self-pollinated to the F₄ or F₅ generation.
^{v}Mean ratings of individual progeny scored for resistance to Phytophthora infestans based on 1-5 (detached leaf ratings) or 0-5 (field ratings) scale (0 or 1 for no lesions, and 5 for completely covered with lesions or dead).
^{u}Individual progeny were rated for resistance to TSWV as R or S and the percentage of susceptibility (S) progenies was calculated.
^{t}H = heterozygous.
^{s}T = homozygous for the TSWV resistant parent NC946 allele (contains the Sw-5 gene).
^{r}P = homozygous for the Phytophthora infestans resistant parent NC592 allele (contains the Ph-3 gene).
^{q}Resistant controls were NC 2CELBR (Ph-3/Ph-3) and 89R (Sw-5/Sw-5).
^{p}Susceptible controls were NC 30P (Ph-3⁺/Ph-3⁺) and 89S (Sw-5⁺/Sw-5⁺).
^{o}P value from analysis of variance for the test of the main effect of entries (F_{3:4} families).

Evaluation of disease resistance. Resistance to TSWV was evaluated on seedlings under greenhouse conditions as previously described [Gordillo, L. F., M. R. Stevens, M. A. Millard, and B. Geary. 2008. Screening two Lycopersicon peruvianum collections for resistance to Tomato spotted wilt virus. Plant Dis. 92:694-704; Stevens, M. R., S. J. Scott, and R. C. Gergerich. 1992. Inheritance of a gene for resistance to Tomato spotted wilt virus (TSWV) from Lycopersicon peruvianum Mill. Euphytica 59:9-17, incorporated by reference herein in their entireties]. Briefly, mechanical inoculation of the “HR-1” isolate was performed at least twice, one week apart. HR-1 is an isolate derived from TSWV infected field tomatoes. This isolate readily infects Sw-5+/Sw-5+ (+/+) lines but does not infect Sw-5/Sw-5 or Sw-5/Sw-5+ genotypes. Near isogenic tomato lines 89R (Sw-5/Sw-5; resistant) and 89S (Sw-5+/Sw-5+; susceptible) were used as controls. Plants with stunted growth, necrotic patches, or chlorotic ringspots were considered infected and scored as homozygous susceptible (Sw-5+/Sw-5+). Individual progeny were rated for resistance to TSWV as R (resistant) or S (susceptible) and the percentage of susceptible (S) progenies was calculated. Two separate evaluations were performed in 2008 and 2010 using different recombinant families (last two right-hand columns of Table 1).

Late blight resistance was evaluated using a detached leaf assay and field tests. Lines NC 2CELBR (Ph-3/Ph-3; resistant) and NC 30P (Ph-3+/Ph-3+; susceptible) were used as controls. Fully expanded young leaves with five leaflets were excised from each of six plants per line using a single-edge razor blade from greenhouse grown plants. Each leaf was placed into 120 mL distilled water in a snap seal plastic sample container with a 1 cm hole in the center of the lid. Containers were placed in a clear plastic box and hand sprayed to atomize a sporangia suspension (5,000 sporangia per mL of distilled water) of Phytophthora infestans isolate 97-1-1 until the upper surface of each excised tomato leaf was completely covered. The boxes were closed and placed in an incubator at 20/16° C. for 12 hour photoperiods. Leaves of each line were evaluated after one week using a score from 1-5 (1 for no lesions, and 5 for completely covered with lesions or dead). These results are shown in the “Detached leaf” column of Table 1. Fifteen resistant lines identified from the detached leaf tests were planted in the field in the summer in two locations (Mills River and Waynesville, N.C.) under natural inoculum without any fungicide spray. Individual plants were scored from 0-5 (0 for no lesions, and 5 for completely covered with lesions or dead). These results are shown in the “Field” column of Table 1.

Molecular marker genotyping. Marker-assisted selection for Ph-3 and Sw-5 was accomplished using previously identified markers. Although in no way limiting, the marker assays described herein are CAPS and SCAR-type assays.

Ph-3 was indirectly selected by a CAPS assay using the cleaved amplified polymorphic sequences (CAPS) markers TG328 (http://solgenomics.net/search/markers/markerinfo.pl?marker_id=171 verified Jun. 23, 2010) and TG591 (verified Jun. 23, 2010). The clone of TG328 has a forward sequence of:

[SEQ. ID. NO. 1]
TGTAGTATTCTAGTTAAACTACCTTTGAATGTCTAGTACCAGACTTA
TAATAGTATTTTGGTAGAATGTCTGCGTGTATATTTAGTTGGGTGAA
TGGACTGGTGATCTGCTTATAGACTTGGGGAATCTTCCCCTCGACCC
CTCACGAGCTAGCTTTTGGGGTTGCATATCATTGTCCAGCTAACCAA
ACTAAACTATATGTATTTAAACCATATATGAAGTATACCTTATGGGA
AATTTGAAGGAATGAAGGTTATTACTAGTATGTTAAACTCATGCTTA
TGTAACTTTGAGCATTAACACATAAAAAGGGGCAGCGCGGTGCACTA
TATTCTCCGGCTATAACTAGGGACATTGAACTTGCATTTTTGCGAGA
GACTATTTAAATGGCTAGAGACAAAGGCACGTTGCCATAATGGACAT
AAATTAACGACTATATACAAAGTTAGCCATGTGTCACGA,
and a reverse sequence of:
[SEQ. ID. NO. 2]
TTTTTTTTATTTGTTGGAAAGAATTGGCTTTTGAATATCAAGAATTA
TTATCTTATTATTATTCCTCTCCATTTTTAATTAAATGGAGGGGGTA
TCTAATAATGTTCATGGAGTTATATTAGCAATATCATCCAGTATTTT
TATTGGTTCCAGCTTTGTTATTAAAAAACAAGGTCTAAAGAAGGCTG
GTGCAAATGGAAAAAGAGCAGGTTTATTCTTTTTCTCGCGCTATGAT
TCAAAAACTCGATGAATAATAGGCTTGCATCTTTCTCCCCTTAAATA
CCAGACTTTTGTATTCAGAGAATCGTGACACATGGCTAACTTTGTAT
ATAGTCGTTAATTTATGTCCATTATGGCAACGTGCCTTTGTCTCTAG
CCATTTAAATAGTCTCTCGCAAAAATGCAAGTTCAATGTCCCTAGTT
ATAGCCGGAGAATATAGTGCACCGCGCTGCCCCTTTTTATGTGTTAA
TG,
and the clone of TG591 has a sequence of:
[SEQ. ID. NO. 3]
TAAATCTGTTGAAATAGCTGCAAATCTACTCGTGCAAGAAGGTACGC
GTTTACATTGGTTGAGGGAGGACATCTAGAGAAATGAGACACATTCG
ATCGTATGTATACGATGCAAAGGCAAAGGAAGTTGGAGGTCAAAAAC
CTATTACAAGATATTCAACAACTGGCAGGTGATGTGGAGGATCTATT
AGATGAGTTCCTTCCAAAAATTCAACAATCCAATAAGGTCATTTGAT
GCCTTAAGACTGTTTCTTTTGCGGATAAGTTTGCTATGGAGATTGAG
AACATAAAAAGAAGAGTTGCTGATATTGACCGTGTAAGGACAACTTA
CCACATCACGGATACTAGTAACAATAATGATGATTGCATTCCAATGG
ACCAGAGAAGACGATTCCTTCATGCTAATGATGAAACAGAGGTCATC
GGATTGGATCATGACTTCAACAAGCTCCAACACAAATTGCTTCTTCA
AGATTTGCCTTACGGAGTTGTTTCA,
and a reverse sequence of:
[SEQ. ID. NO. 4]
GGAGACAGCTTGCATGCCTGCAGTCACCACAATCGCTAGCGGTATAC
CTCCACATCTCTCAACTATACATCTACCAATATTTACCAAGTCTGGT
GAAGTATTGGCCCAATTATCATCAACAAAATTACAGATTTTCTTGGT
AAAGAGTTCAAAACTGTTCTCTGAGTCTAGAGGTTGCAACTCGTGGA
TTGAGAAATCCCCTCCTATGTATCTGCCTACATTACTATTTCGAGAG
GTTATAATTATCCTACTGCCAATTTTTGAATACGATTCAGGAAGGAC
GAGTTTTAGATCATCCCAAATTTCAACATCCCAAATGTCATCTAAAA
GGATAACGTACCTTTTTATTTTCAAGAGTGATCTTAGGTTGTGTTCC
AAGTTCTCTTTCCTTTCTTCTTCCGTCAGTCCAACTTGTTTGGCTAT
GTTAAGCAAGATTTCTCCCGCCCTTGGCTGCTGCGAGACATAGACCA
ATCCAGAACATTCAAATTGATGACGGACGTGCCTATAAAGTTTCTTG
GCAAGAGTAGTTTTTCCCAAACCGGG.

TG328 was detected using the primers (5′ to 3′) TG328F (GGTGATCTGCTTATAGACTTGGG) [SEQ. ID. NO. 5] and TG328R (AAGGTCTAAAGAAGGCTGGTGC) [SEQ. ID. NO. 6] and the restriction endonuclease BstNI. For TG591, the primers TG591 F (AAGGCAAAGGAAGTTGGAGGTCA) [SEQ. ID. NO.7] and TG591 R (AGAGGTTGCAACTCGTGGATTGAG) [SEQ. ID. NO. 8] were employed with AciI. Fragment sizes for TG328:BstNI were 500 by (undigested) for the NC946 allele (T) and a 260/240 by doublet for the NC592 allele (P). For the TG591:AciI digest, a multiple band phenotype was observed with the diagnostic polymorphism as a 150 by fragment for the NC946 allele (T) and a 160 fragment for the NC592 allele (P).

To indirectly select for Sw-5, a SCAR assay was used, and the sequenced characterized amplified region (SCAR) marker SCAR421 was utilized [see Anfoka, G. H., M. Abhary, and M. R. Stevens. 2006. Occurrence of Tomato spotted wilt virus (TSWV) in Jordan. EPPO Bulletin 36:517-522; Folkertsma, R. T., M. I. Spassova, M. Prins, M. R. Stevens, J. Hille, and R. W. Goldbach. 1999. Construction of a bacterial artificial chromosome (BAC) library of Lycopersicon esculentum cv. Stevens and its application to physically map the Sw-5 locus. Mol. Breed. 5:197-207; Stevens, M. R., D. K. Heiny, P. D. Griffiths, J. W. Scott, and D. D. Rhoads. 1996. Identification of co-dominant RAPD markers tightly linked to the Tomato spotted wilt virus (TSWV) resistance gene Sw-5. Rep. Tomato Genet. Coop. 46:27-28, the disclosures of which are incorporated by reference herein in their entireties]. SCAR421 amplified a 940 by fragment in NC946 (T) and a 920 by fragment in NC592 (P). The primers for amplifying SCAR421 were 421-1 GACTTGTTGCCATAGGTTCC [SEQ. ID. NO. 9] and 421-2 GCCCACCCCGAAGTTAATCC [SEQ. ID. NO. 10]. For all populations evaluated with markers, all individuals were initially genotyped, then all selections were subsequently resampled and regenotyped for confirmation.

Like many other molecular marker tests, the CAPS and SCAR assays use the polymerase chain reaction, or PCR, which can amplify DNA from very small amounts of starting material. The use of PCR is well known to those skilled in the art, and allows the researcher to only harvest very small amounts of plant sample in order to perform the test. For example, less than 1 cm² of leaf material, preferably young actively growing tissue, is needed to perform these tests. This is such a small sample that these marker tests are usually referred to as non-destructive. They are considered non-destructive as the taking of the sample does not interfere whatsoever in the way the plant develops. Thus, this small sample does not affect the outcome of any number of subsequent tests, from pathology testing, fruit biochemical analysis, yield trialing, or horticultural evaluations. In addition to being non-destructive, the CAPS and SCAR assays also provide the additional advantage of time. Typically, the genotype at the locus or loci of interest can be ascertained within 24 hours.

The molecular marker tests used begin with the isolation of genomic DNA. In this Example, genomic DNA was isolated in 96-well format following the modified CTAB method described in Kabelka, E., B. Franchino, and D. M. Francis, 2002, Two loci from Lycopersicon hirsutum LA407 confer resistance to strains of Clavibacter michiganensis subsp. michiganensis. Phytopathology 92:504-510, incorporated by reference herein in its entirety. However, those of ordinary skill in the art will recognize that there are other methods to isolate genomic DNA. For example, although no way limiting, the following protocol could alternatively be used to extract tomato DNA for the subsequent molecular marker testing:

1. Collect a plant part that is approximately the size of a well in the 96 well microtiter plate format. Preferably, a tissue sample is taken from young leaves, though a seed sample or other plant tissues are possible sources of DNA.

2. Add 150 μl extraction buffer (200 mM Tris-HCl, pH 7.5; 250 mM NaCl; 25 mM EDTA; 0.5% SDS) to the sample and macerate the tissue.

3. Centrifuge the plate for 15 minutes at 1900^{x}g at 15° C.

4. Transfer 100 μl of the supernatant fraction to a new 96 well plate that contains 100 μl of 2.5M potassium acetate (pH 6.5) in each well. Mix by shaking for approximately 2 minutes at 200 rpm.

5. Centrifuge the plate for 15 minutes at 1900^{x}g at 15° C.

6. Transfer 75 μl of the supernatant fraction to a new 96 well plate containing 75 μl isopropanol. Mix, then shake for 2 minutes at 200 rpm.

7. Centrifuge the plate for 15 minutes at 1900^{x}g at 15° C.

8. Remove supernatant fraction and add 200 μl 70% ethanol to the pellet fraction. Shake at 200 rpm for 5 minutes, and then incubate overnight at −20° C.

9. Centrifuge the plate for 15 minutes at 1900^{x}g at 15° C.

10. Remove supernatant fraction. Add 200 μl of 70% ethanol to the pellet, allowing the alcohol to wash the pellet for 1 hour at room temperature.

11. Centrifuge the plate for 15 minutes at 1900^{x}g at 15° C.

12. Discard the supernatant fraction and dry the pellet fraction at room temperature. This takes about 1 hour.

13. Dissolve the pellet fraction in 100 μl TE (10 mM Tris, pH 8.0, 1 mM EDTA, 5 μg/ml RNAase A) for 15 minutes at 37° C. Unless proceeding to the PCR step, the DNA can be stored at 4° C. or −20° C.

General PCR Conditions for the CAPS and SCAR Assays. The primers used in the CAPS and SCAR assays are described above; these stretches of nucleotides match the DNA sequence of the substrate at the marker locus to be amplified. The primers are designed such that they will facilitate the synthesis of an amplicon typically between 100 to 1,000 base pairs. Those skilled in the art know how to design these primers to create CAPS and SCAR-type assays, and often use software programs, like the publicly available Primer3 software (Whitehead Institute, Cambridge, Mass.) to assist in the design. Primers can be synthesized using methodology known in the art, or purchased from any number of custom oligonucleotide companies. All primers used in these assays were purchased from Eurofins mwg/Operon (Huntsville, Ala.). Other reagents in the PCR reaction can be purchased from any number of commercial suppliers; in the assays described herein, we purchased Taq polymerase for PCR from Fisher Scientific (Pittsburgh, Pa.); nucleotides for PCR from Promega Corporation (Madison, Wis.); Restriction Endonucleases for CAP assays from Fermentas Life Sciences, (Glen Burnie, Md.) or New England Biolabs (Ipswich, Mass.);. Those skilled in the art recognize that there is some flexibility in performing CAP, SCAR, ASPE and other nucleotide polymorphism detection assays because there are many types of PCR machines and assay conditions that can be used. A DNA Engine Tetrad2 Peltier Thermal Cycler (BioRad, Hercules, Calif.) PCR machine was used with the run parameters described below for each of the assays.

To determine marker genotypes, genomic DNA was isolated in 96-well format following the modified CTAB method described in Kabelka, E., B. Franchino, and D. M. Francis, 2002, Two loci from Lycopersicon hirsutum LA407 confer resistance to strains of Clavibacter michiganensis subsp. michiganensis. Phytopathology 92:504-510, incorporated by reference herein in its entirety, and subjected to PCR. Conditions for PCR reactions were 10 mM Tris-HCl (pH 8.3), 50 mM KCl, 1.5 mM MgCl2, 50 μM of each dNTP, 0.1 μM of each forward and reverse primers, 20 ng of template DNA, and 1 unit of Taq DNA polymerase in a total volume of 10-20 μl. PCR amplification was performed using the cycling parameters of 30 s at 94° C. followed by 35 cycles of 20 s at 94° C., 20 s at 55° C., and 2 min at 72° C., followed by an extended incubation for 7 min at 72° C. Those skilled in the art recognize that there is considerable flexibility allowed in the PCR assay conditions.

For CAPS markers, PCR amplicons were digested with the appropriate restriction endonuclease by adding 0.08-0.16 units of enzyme in 1× digestion buffer for each μl of PCR product. Markers were visualized as size polymorphisms by agarose gel electrophoresis. Genotype frequencies for all markers were tested for deviation from expected segregation ratios by a χ2 goodness of fit test. Map distances between markers were calculated from the genotypic data of the original F2 population using JoinMap® 3.0 [Van Ooijen, J. W. and R. E. Voorrips. 2001. JoinMap® 3.0, software for the calculation of genetic linkage maps. Plant Research International, Wageningen, The Netherlands]. The markers were first grouped with a LOD of at least 10.0, then the Kosambi mapping function was utilized to calculate the distance between markers in cM.

Each SCAR assay also has commonality in how the polymorphisms are revealed after the PCR reactions have been completed. For each test, the amplified region of DNA from the PCR reaction may contain a polymorphic restriction enzyme recognition site. Alternate alleles either have, or do not have this recognition site. When the PCR products are digested with a specific restriction enzyme, the amplicon is either not cut because the restriction enzyme site is not present, or digested asymmetrically into two fragments. The genotype of the locus can be determined by electrophoretically resolving these fragments on an agarose gel, staining the gel with ethidium bromide, which is a stain that binds DNA, then visualizing the fragments by exciting the DNA with ultraviolet light. The fragment sizes from the PCR reactions are determined by comparing them to known size standards, which are electrophoresed in nearby lanes in the agarose gel.

### Results and Discussion

Selection of coupling phase recombinants using molecular markers. A total of 1152 F₂ plants derived from parents NC946 and NC592 were subjected to DNA marker analysis using SNPs in genomic DNA amplicons defined by CAPS markers TG328 and TG591 and an insertion/deletion detected by PCR marker SCAR421. Of the 1152 F₂ plants, 24 were identified as recombinants with 11 potentially having both resistances in coupling phase. Of these 11 recombinants, three were male sterile. The remaining 8 F₂ recombinants are shown in the left-hand column of Table 1 (08-5401, 08-5402, 08-5403, 08-5404, 08-5405, -8-5406, -8-5407, and 08-5408).

The observed recombination events suggested a marker order of TG328, TG591, and SCAR421, consistent with previous maps and the physical order of the markers on bacterial artificial chromosome (BAC) clones for chromosome 9 [Folkertsma, R. T., M. I. Spassova, M. Prins, M. R. Stevens, J. Hille, and R. W. Goldbach. 1999. Construction of a bacterial artificial chromosome (BAC) library of Lycopersicon esculentum cv. Stevens and its application to physically map the Sw-5 locus. Mol. Breed. 5:197-207]. However, recombination distances of 0.33 cM between TG328 and TG591 and 0.78 cM between TG591 and SCAR421 suggest that recombination was suppressed between the S. peruvianum introgression (Sw-5) and the S. pimpinellifolium introgression (Ph-3) segments. In contrast, markers TG328 and TG591 are separated by 1 cM on the reference maps while TG591 and SCAR421 (physically linked to CT220) are separated by 5.0 cM [Pillen, K., O. Pineda, C. B. Lewis, and S. D. Tanksley. 1996. Status of genome mapping tools in the taxon Solonaceae, p. 281-308. In: Paterson, A. H. (ed.), Genome Mapping in Plants. Academic Press, Austin, Tex.; Tanksley, S. D., M. W. Ganal, J. P. Prince, M. C. de Vicente, M. W. Bonierbale, P. Broun, T. M. Fulton, J. J. Giovannoni, S. Grandillo, G. B. Martin, R. Messeguer, J. C. Miller, L. Miller, A. H. Paterson, O. Pineda, M. S. Roder, R. A. Wing, W. Wu, and N. D. Young. 1992. High density molecular linkage maps of the tomato and potato genomes. Genetics 132:1141-1160] on the EXPEN 2000 map (http://www.sgn.cornell.edu). Suppression of recombination is not unexpected in introgressions [Ganal, M. W. and S. D. Tanksley. 1996. Recombination around the Tm2a and Mi resistance genes in different crosses of Lycopersicon peruvianum. Theor. Appl. Genet. 92:101-108; Liharska, T., M. Koornneef, M. vanWordragen, A. vanKammen, and P. Zabel. 1996. Tomato chromosome 6: Effect of alien chromosomal segments on recombinant frequencies. Genome 39:485-491; Zamir, D. and Y. Tadmor. 1986. Unequal segregation of nuclear genes in plants. Bot. Gaz. 147:355-358].

The eight recombinant F₂ individuals (shown in Table 1) were retained, and self-pollinated to produce F₃ families. For each of the eight recombinant F₂ plants which produced seed in the greenhouse, 40 individual F₃ plants were transplanted for evaluation in the field. DNA extracted from the 320 (8×40) F₃ plants was genotyped and 65 F₃ individuals that were homozygous for the desired recombination events were identified. Thirty-five F₃ individuals were eliminated due to undesirable vine and/or fruit characteristics and 30 plants were retained based on desirable characteristics of vine type, heavy fruit load, uniform fruit color, fruit shape and fruit size based on visual assessment. Among the 30 selected F₃ plants were representatives from each of the eight recombinant F₂ plants.

Confirmation of resistances of F4 progeny. For phenotypic confirmation, F_{3:4} progenies derived from each of the eight F₂ coupling phase recombinants, were challenged with P. infestans and TSWV. Two F₄ families from each F₂ were evaluated in 2008, and a replicate evaluation using related families (either F₄ or F₅) were tested during the summer 2009 season or in the greenhouse in 2010. As can be seen from the data in Table 1, individual families derived from 08-5401, 08-5404, and 08-5406 were significantly more susceptible to P. infestans than the resistant control. These families were more resistant than susceptible controls; thus the phenotype was considered intermediate and ambiguous. At least five of the F_{2:4} families derived from independent F₂ recombinants were unambiguously resistant to late blight. Families derived from 08-5402 appeared to segregate or show less than complete resistance to TSWV while families derived from 08-5407 were fully susceptible. Families derived from, 08-5403, 08-5405 and 08-5408 showed resistance to both diseases (Table 1). Additionally, F₅ families were tested for confirmation of the original F₂ recombinants 08-5401, 08-5403, and 08-5408.

Genetic and physical order of the Ph-3 and Sw-5 genes. Based on available genetic and physical data it appears that the members of the Sw-5 family responsible for TSWV resistance lie within the interval defined by TG591 and CT202, with SCAR421 distal [Brommonschenkel, S. H., A. Frary, and S. D. Tanksley. 2000. The broad-spectrum tospovirus resistance gene Sw-5 of tomato is a homolog of the root-knot nematode resistance gene Mi. Mol. Plant-Microbe Interact. 13:1130-1138; Folkertsma, R. T., M. I. Spassova, M. Prins, M. R. Stevens, J. Hille, and R. W. Goldbach. 1999. Construction of a bacterial artificial chromosome (BAC) library of Lycopersicon esculentum cv. Stevens and its application to physically map the Sw-5 locus. Mol. Breed. 5:197-207). The data herein suggests that the likely position for Ph-3 is between TG328 and TG591. The occurrence of several resistance gene homologs on BAC clones containing TG591 suggests possible candidates. Based on the length of the sequenced BAC clones, it appears that the position of Ph-3 is likely to be physically separated from Sw-5 by over 100,000 by despite the low genetic distance separating the loci in the NC592 by NC946 cross.

The marker and resistance gene order suggested by recombinant families is consistent with the physical organization of the Sw-5 region of chromosome 9. Sequence data for BACs is available from [Folkertsma, R. T., M. I. Spassova, M. Prins, M. R. Stevens, J. Hille, and R. W. Goldbach. 1999. Construction of a bacterial artificial chromosome (BAC) library of Lycopersicon esculentum cv. Stevens and its application to physically map the Sw-5 locus. Mol. Breed. 5:197-207] and from the international tomato genome sequencing effort. Marker TG328 is not yet anchored to a BAC. Marker TG591 is anchored to a 110,571 by BAC clone, C09Hba0165P17 (GenBank EF647605 GI:149930469). BAC clones containing Sw-5 family members Sw-5a (AY007366) and Sw-5c (AY007367) have been sequenced [Folkertsma, R. T., M. I. Spassova, M. Prins, M. R. Stevens, J. Hille, and R. W. Goldbach. 1999. Construction of a bacterial artificial chromosome (BAC) library of Lycopersicon esculentum cv. Stevens and its application to physically map the Sw-5 locus. Mol. Breed. 5:197-207]. These include a 35,250 by BAC containing the Sw-5 locus (GenBank AY007366; GI:15418708) with significant homology to the 114,526 by BAC, C09HBa109D11 (GenBank EF647603; GI:149930467), and the 113,581 by BAC clone C09Hba226D21 (GenBank EU139072; GI:157649058). The 3′ end of C09Hba109D11 overlaps with the 5′ end of clone C09Hba226D21 suggesting a possible contiguous sequence. CT202 is anchored to a 95,676 by BAC Hba0059I05 (SGN only, as this sequence was not yet deposited in GenBank at the time of this writing). The marker SCAR421 is physically linked to CT202 within about 65 Kb (kilobases) [Folkertsma, R. T., M. I. Spassova, M. Prins, M. R. Stevens, J. Hille, and R. W. Goldbach. 1999. Construction of a bacterial artificial chromosome (BAC) library of Lycopersicon esculentum cv. Stevens and its application to physically map the Sw-5 locus. Mol. Breed. 5:197-207]. The 5′ region of BAC Hba0059I05 has homology to C09Hba109D11 and homology to the Sw-5a protein. The 3′ end of BAC Hba0059I05 95676 has no significant homology to sequences in the NCBI database.

The embodiments of the present invention recited herein are intended to be merely exemplary and those skilled in the art will be able to make numerous variations and modifications to it without departing from the spirit of the present invention. Notwithstanding the above, certain variations and modifications, while producing less than optimal results, may still produce satisfactory results. All such variations and modifications are intended to be within the scope of the present invention as defined by the claims appended hereto.

What is claimed is :

1. A Solanum lycopersicum plant comprising within its genome at least one Tomato Spotted Wilt Virus (TSWV) resistance allele and at least one Phytophthora infestans resistance allele, wherein said resistance alleles are present in the coupling phase at different loci on one chromosome and said plant is resistant against TSWV and resistant against at least Phytophthora infestans, and wherein said at least one Phytophthora infestans resistance allele is an allele designated as Ph-3.

2. The plant of claim 1 having a Phytophthora infestans resistance score of less than about 1.25 in a detached leaf assay or 1.5 under a field evaluation.

3. The plant of claim 1 having Phytophthora infestans resistance score of less than about 2 in a detached leaf assay or 2.5 under a field evaluation.

4. The plant of claim 1 or plurality of plants derived from the plant of claim 1, having TSWV resistance score of less than about 10%.

5. The plant of claim 1, wherein the TSWV resistance allele is the allele designated as Sw-5.

6. The plant according to claim 1 wherein said chromosome is chromosome 9.

7. The plant according to claim 1 wherein said TSWV resistance allele and said Phytophthora infestans resistance allele are non-transgenic.

8. The plant according to claim 1, wherein said TSWV resistance allele and said Phytophthora infestans resistance allele are from Sw. peruvianum and from S. pimpinellifolium, respectively.

9. A fruit or a seed of the plant of claim 1.

10. A hybrid Solanum lycopersicum plant produced by the method of crossing the plant in claim 1 with an inbred plant lacking said TSWV resistance allele and lacking said Phytophthora infestans resistance allele.

11. A hybrid Solanum lycopersicum plant of claim 1, wherein both of said TSWV resistance allele and said Phytophthora infestans resistance allele are heterozygous.

12. The plant of claim 11, wherein the loci to said TSWV resistance allele and said Phytophthora infestans resistance allele occur within the same disease resistance cluster on said chromosome.

13. The plant of claim 12, comprising at least one additional disease resistance allele within said cluster, wherein said additional disease resistance allele is located on the chromosome in trans to said chromosome having said TSWV resistance allele and said Phytophthora infestans resistance allele.

14. The plant of claim 13, wherein said additional disease resistance allele provides resistance to a disease chosen from Bacterial Wilt, Bacterial Canker, Bacterial Speck, Bacterial Spot, Fusarium Crown Rot, Alternaria Stem Canker, Alternaria Collar Rot, Early Blight, Leaf Mold, Fusarium Wilt, Powdery Mildew, Corky Root, Stemphylium Leaf Blight, Septoria Leaf Spot, Verticillium Wilt, Potato Cyst Nematode, Greenhouse Whitefly, Root Knot Nematode, Late Blight, Broomrape, and Alfalfa Mosaic Virus.

15. The plant of claim 1 wherein said plant is an inbred Solanum lycopersicum L.

16. A Solanum lycopersicum plant comprising within its genome at least one Tomato Spotted Wilt Virus (TSWV) resistance allele and at least one Phytophthora infestans resistance allele, wherein said resistance alleles are co-inherited on one chromosome and said plant is resistant against TSWV and highly resistant or moderately resistant against Phytophthora infestans, wherein said at least one Phytophthora infestans resistance allele is the Ph-3 resistance allele.

17. The plant according to claim 16, wherein said plant has a Phytophthora infestans resistance score of less than about 1.25 in a detached leaf assay or 1.5 under a field evaluation.

18. The plant according to claim 16, wherein said TSWV resistance allele is the allele designated as Sw-5.

19. A method for producing a hybrid Solanum lycopersicum plant, said method comprising: (a) obtaining a first Solanum lycopersicum plant of claim 1; and (b) crossing said first Solanum lycopersicum plant with a second Solanum lycopersicum plant bearing an additional resistance allele; wherein said hybrid Solanum lycopersicum plant has two resistance alleles present in the coupling phase.

20. The method of claim 19, wherein said two resistance alleles present in the coupling phase are in trans with said additional resistance allele.

21. The method of claim 19, wherein said two resistance alleles present in the coupling phase are located on chromosome 9.

22. The method of claim 19, wherein one of said two resistance alleles present in the coupling phase is a TSWV resistance allele.

23. The method of claim 19, wherein one of said two resistance alleles present in the coupling phase is Ph-3.

24. A Solanum lycopersicum plant comprising within its genome at least one Sw-5 Tomato Spotted Wilt Virus (TSWV) resistance allele and at least one Ph-3 Phytophthora infestans resistance allele, wherein the at least one Sw-5 allele and the at least one Ph-3 allele are present in the coupling phase at different loci on one chromosome and said plant is resistant against TSWV and highly resistant or moderately resistant against Phytophthora infestans.

25. A fruit or a seed of the plant of claim 15.

26. A fruit or a seed of the plant of claim 16.

27. A fruit or a seed of the plant of claim 24.

28. A Solanum lycopersicum plant comprising within its genome at least one Tomato Spotted Wilt Virus (TSWV) resistance allele and at least one Phytophthora infestans resistance allele, wherein said resistance alleles are present in the coupling phase at different loci on one chromosome and said plant is resistant against TSWV and resistant against at least Phytophthora infestans, and wherein said at least one TSWV resistance allele is an allele designated as Sw-5.

29. A method for producing a hybrid Solanum lycopersicum plant, said method comprising: (a) obtaining a first Solanum lycopersicum plant of claim 28; and (b) crossing said first Solanum lycopersicum plant with a second Solanum lycopersicum plant bearing an additional resistance allele; wherein said hybrid Solanum lycopersicum plant has two resistance alleles present in the coupling phase.
